(19) United States
(12) Patent Application Publication
(10) Pub. No.: US 2022/0233721 A1
(43) Pub. Date: Jul. 28, 2022

(54) METHOD FOR TREATING MUSCULAR DYSTROPHY BY TARGETING DMPK GENE

(71) Applicants: Astellas Pharma Inc., Chuo-ku (JP); Modalis Therapeutics Corporation, Tokyo (JP)

(72) Inventors: Eiji YOSHIMI, Chuo-ku (JP); Tomoya OE, Chuo-ku (JP); Tetsuya YAMAGATA, Cambridge, MA (US); Keith M. CONNOLLY, Cambridge, MA (US)

(73) Assignees: Astellas Pharma Inc., Chuo-ku (JP); Modalis Therapeutics Corporation, Chuo-ku (JP)

(21) Appl. No.: 17/614,461

(22) PCT Filed: May 27, 2020
(86) PCT No.: PCT/JP2020/021851

§ 371 Date: Nov. 26, 2021

Related U.S. Application Data
(60) Provisional application No. 63/025,417, filed on May 15, 2020.
(60) Provisional application No. 62/853,373, filed on May 28, 2019.

(51) Int. Cl. A61K 48/00; A61K 35/76; A61K 38/46; A61K 31/7088

(52) U.S. Cl. CPC A61K 48/0058; A61K 48/0066; A61K 31/7088; A61K 38/465; A61K 35/76

(57) ABSTRACT

Polynucleotides comprising the following base sequences: (a) a base sequence encoding a fusion protein of a nuclease-deficient CRISPR effector protein and a transcriptional repressor, and (b) a base sequence encoding a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in an expression regulatory region of a human DMPK gene, are expected to be useful for treating muscular dystrophy.

### CROSS REFERENCE TO RELATED APPLICATIONS

This application claims the benefit of U.S. Provisional Patent Application No. 62/853,373, filed on May 28, 2019, and U.S. Provisional Patent Application No. 63/025,417, filed on May 15, 2020, the contents of which are incorporated herein by reference in their entireties.

### BACKGROUND OF THE INVENTION

### Field of the Invention

The present invention relates to methods for treating muscular dystrophy by targeting the human myotonin protein kinase (DMPK; dystrophia myotonica protein kinase) gene and the like. More particularly, the present invention relates to methods and pharmaceutical compositions for treating or preventing muscular dystrophy by repressing expression of human DMPK gene by using a guide RNA targeting a particular sequence of human DMPK gene and a fusion protein of a transcriptional repressor and a CRISPR (clustered regularly interspaced short palindromic repeat) effector protein, and the like.

### Discussion of the Background

Muscular dystrophy is a generic term for hereditary diseases associated with progressive muscular atrophy and muscle weakness. Even today, a fundamental therapeutic drug effective for muscular dystrophy does not exist, and only symptomatic treatments are performed. Among muscular dystrophies, myotonic dystrophy type 1 (DM1) is caused by mutations in the DMPK gene.

DM1 is an autosomal dominant genetic disease caused by elongation of CTG repeats in the 3′ untranslated region (3′ UTR) of the DMPK gene and is one type of triplet repeat disease. It has been reported that, in DM1, RNA containing an expanded CUG repeat isolates CUG repeat binding proteins such as MBNL (Muscleblind-like) from endogenous RNA targets, thereby causing aberrant splicing patterns, changes in RNA stability/localization, and the like. These findings suggest that silencing of expanded repeat loci has therapeutic value, and various approaches such as antisense oligonucleotide, small RNA, small molecules, and the like are used to silence toxic RNA (see Pinto B et al., Mol Cell. 2017 Nov. 2, 68(3):479-490, which is incorporated herein by reference in its entirety).

For example, Jauvin et al. treated DMSXL mice, which is a mouse model of DM1, with an antisense oligonucleotide (ASO) targeting 3′ UTR of DMPK gene, and showed that the DMPK mRNA level decreased, nuclear RNA aggregates (RNA foci) decreased and muscle strength increased, whereas no apparent toxicity was detected (see Jauvin D et al., Mol Ther Nucleic Acids. 2017 Jun. 16, 7:465-474, which is incorporated herein by reference in its entirety).

WO2018/002812 discloses a method for editing a DMPK gene in a cell by genome editing, e.g. using the CRISPR/Cas9 system, which can be used to treat a DMPK related condition or disorder such as DM1 (see WO2018/002812, which is incorporated herein by reference in its entirety).

Pinto et al. and Batra et al. demonstrated the possibility of the application of deactivated/nuclease-dead Cas9 (dCas9) to the treatment of DM1. To be specific, Pinto et al. combined dCas9 and gRNA to CTG repeat region and showed that dCas9 effectively blocks transcription of expanded microsatellite repeat, whereby the phenotypes characteristic of DM1, which are due to repeat expansion, can be improved in vitro and in vivo (in HSA^{LR}mouse which is a mouse model of DM1) (see Pinto B et al., Mol Cell. 2017 Nov. 2, 68(3):479-490, which is incorporated herein by reference in its entirety). On the other hand, Batra et al. showed that a combination of dCas9 fused with RNA endonuclease and gRNA for the CUG repeat region of DMPK mRNA can reduce level of CUG repeat expansion RNA and improve splicing abnormality in the cells of DM1 patients (see Batra R et al., Cell. 2017 Aug. 24, 170(5):899-912, which is incorporated herein by reference in its entirety).

### SUMMARY OF THE INVENTION

Accordingly, it is one object of the present invention is to provide novel therapeutic methods to muscular dystrophy (particularly DM1).

It is another object of the present invention to provide novel agents which are useful for treating muscular dystrophy.

These and other objects, which will become apparent during the following detailed description, have been achieved by the inventors' discovery that the expression of human DMPK gene can be strongly suppressed using a guide RNA targeting a particular sequence of human DMPK gene (Gene ID: 1760) and a fusion protein of a transcriptional repressor and a nuclease-deficient CRISPR effector protein. Based on these findings, the present inventors have completed the present invention.

Thus, the present invention provides the following:

(1) A polynucleotide comprising the following base sequences:

(a) a base sequence encoding a fusion protein of a nuclease-deficient CRISPR effector protein and a transcriptional repressor, and

(b) a base sequence encoding a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene.

(2) The polynucleotide of the above-mentioned (1), comprising the following base sequences:

(a) a base sequence encoding a fusion protein of a nuclease-deficient CRISPR effector protein and a transcriptional repressor, and

(b) a base sequence encoding a guide RNA targeting a continuous region of 18 to 24 nucleotides in length in a region set forth in SEQ ID NO: 127, SEQ ID NO: 46, SEQ ID NO: 128, SEQ ID NO: 66, SEQ ID NO: 68, SEQ ID NO: 130, SEQ ID NO: 131, SEQ ID NO: 132, SEQ ID NO: 88, SEQ ID NO: 91, SEQ ID NO: 96, SEQ ID NO: 99, SEQ ID NO: 100, SEQ ID NO: 134, SEQ ID NO: 108, SEQ ID NO: 109, SEQ ID NO: 111, SEQ ID NO: 117, or SEQ ID NO: 119 in the expression regulatory region of human DMPK gene.

(3) The polynucleotide of the above-mentioned (1) or (2), comprising the following base sequences:

(a) a base sequence encoding a fusion protein of a nuclease-deficient CRISPR effector protein and a transcriptional repressor, and

(b) a base sequence encoding a guide RNA targeting a continuous region of 18 to 24 nucleotides in length in a region set forth in SEQ ID NO: 63, SEQ ID NO: 136, SEQ ID NO: 83, SEQ ID NO: 99, SEQ ID NO: 135, SEQ ID NO: 109, or SEQ ID NO: 111 in the expression regulatory region of human DMPK gene.

(4) The polynucleotide of the above-mentioned (1), wherein the base sequence encoding the guide RNA comprises the base sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117, or SEQ ID NO: 119, or the base sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117, or SEQ ID NO: 119 in which 1 to 3 bases are deleted, substituted, inserted, and/or added.

(5) The polynucleotide of any of the above-mentioned (1) to (4), comprising at least two base sequences encoding the guide RNAs, wherein the at least two base sequences are different.

(6) The polynucleotide of any of the above-mentioned (1) to (5), wherein the transcriptional repressor is selected from the group KRAB, MeCP2, SIN3A, HDT1, MBD2B, NIPP1, and HP1A.

(7) The polynucleotide of the above-mentioned (6), wherein the transcriptional repressor is KRAB.

(8) The polynucleotide of any of the above-mentioned (1) to (7), wherein the nuclease-deficient CRISPR effector protein is dCas9.

(9) The polynucleotide of the above-mentioned (8), wherein the dCas9 is derived from Staphylococcus aureus.

(10) The polynucleotide of any of the above-mentioned (1) to (9), further comprising a promoter sequence for the base sequence encoding the guide RNA and/or a promoter sequence for the base sequence encoding the fusion protein of the nuclease-deficient CRISPR effector protein and the transcriptional repressor.

(11) The polynucleotide of the above-mentioned (10), wherein the promoter sequence for the base sequence encoding the guide RNA is selected from the group U6 promoter, SNR6 promoter, SNR52 promoter, SCR1 promoter, RPR1 promoter, U3 promoter, and H1 promoter.

(12) The polynucleotide of the above-mentioned (11), wherein the promoter sequence for the base sequence encoding the guide RNA is U6 promoter.

(13) The polynucleotide of any of the above-mentioned (10) to (12), wherein the promoter sequence for the base sequence encoding the fusion protein of the nuclease-deficient CRISPR effector protein and the transcriptional repressor is a ubiquitous promoter or a muscle specific promoter.

(14) The polynucleotide of the above-mentioned (13), wherein the ubiquitous promoter is selected from the group EFS promoter, CMV promoter and CAG promoter.

(15) The polynucleotide of the above-mentioned (13), wherein the muscle specific promoter is selected from the group CK8 promoter, myosin heavy chain kinase (MHCK) promoter, muscle creatine kinase (MCK) promoter, synthetic C5-12 (Syn) promoter, and Des promoter.

(16) The polynucleotide of the above-mentioned (15), wherein the muscle specific promoter is CK8 promoter.

(17) The polynucleotide of any of the above-mentioned (10) to (16),

wherein the base sequence encoding the guide RNA comprises the base sequence set forth in SEQ ID NO: 70, SEQ ID NO: 81, SEQ ID NO: 83, or SEQ ID NO: 99, or the base sequence set forth in SEQ ID NO: 70, SEQ. ID NO: 81, SEQ ID NO: 83, or SEQ ID NO: 99 in which 1 to 3 bases are deleted, substituted, inserted, and/or added,

the transcriptional repressor is KRAB,

the nuclease-deficient CRISPR effector protein is dCas9 derived from Staphylococcus aureus,

the promoter sequence for the base sequence encoding the guide RNA is U6 promoter, and

the promoter sequence for the base sequence encoding the fusion protein of the nuclease-deficient CRISPR effector protein and the transcriptional repressor is CK8 promoter.

(18) The polynucleotide of the above-mentioned (17),

wherein the base sequence encoding the guide RNA comprises the base sequence set forth in SEQ ID NO: 83, or the base sequence set forth in SEQ ID NO: 83 in which 1 to 3 bases are deleted, substituted, inserted, and/or added.

(19) A vector comprising a polynucleotide of any of the above-mentioned (1) to (18).

(20) The vector of the above-mentioned (19), wherein the vector is a plasmid vector or a viral vector.

(21) The vector of the above-mentioned (20), wherein the viral vector is selected from the group adeno-associated virus (AAV) vector, adenovirus vector, and lentivirus vector.

(22) The vector of the above-mentioned (21), wherein the AAV vector is selected from the group AAV1, AAV2, AAV6, AAV7, AAV8, AAV9, Anc80, AAV587MTP, AAV588MTP, AAV-B1, AAVM41, and AAVrh74.

(23) The vector of (22), wherein the AAV vector is AAV9.

(24) A pharmaceutical composition comprising a polynucleotide of any of the above-mentioned (1) to (18) or a vector of any of the above-mentioned (19) to (23).

(25) The pharmaceutical composition of the above-mentioned (24) for treating or preventing myotonic dystrophy type 1.

(26) A method for treating or preventing myotonic dystrophy type 1, comprising administering a polynucleotide of any of the above-mentioned (1) to (18), or a vector of any of the above-mentioned (19) to (23), to a subject in need thereof.

(27) Use of a polynucleotide of any of the above-mentioned (1) to (18), or a vector of any of the above-mentioned (19) to (23) for the treatment or prevention of myotonic dystrophy type 1.

(28) Use of a polynucleotide of any of the above-mentioned (1) to (18), or a vector of any of the above-mentioned (19) to (23) in the manufacture of a pharmaceutical composition for the treatment or prophylaxis of myotonic dystrophy type 1.

(29) A ribonucleoprotein comprising the following:

(c) a fusion protein of a nuclease-deficient CRISPR effector protein and a transcriptional repressor, and

(d) a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene.

(30) The ribonucleoprotein of the above-mentioned (29), comprising the following:

(c) a fusion protein of a nuclease-deficient CRISPR effector protein and a transcriptional repressor, and

(d) a guide RNA targeting a continuous region of 18 to 24 nucleotides in length in a region set forth in SEQ ID NO: 127, SEQ ID NO: 46, SEQ ID NO: 128, SEQ ID NO: 66, SEQ ID NO: 68, SEQ ID NO: 130, SEQ ID NO: 131, SEQ ID NO: 132, SEQ ID NO: 88, SEQ ID NO: 91, SEQ ID NO: 96, SEQ ID NO: 99, SEQ ID NO: 100, SEQ ID NO: 134, SEQ ID NO: 108, SEQ ID NO: 109, SEQ ID NO: 111, SEQ ID NO: 117, or SEQ ID NO: 119 in the expression regulatory region of human DMPK gene.

(31) The ribonucleoprotein of the above-mentioned (29) or (30), comprising the following:

(c) a fusion protein of a nuclease-deficient CRISPR effector protein and a transcriptional repressor, and

(d) a guide RNA targeting a continuous region of 18 to 24 nucleotides in length in a region set forth in SEQ ID NO: 63, SEQ ID NO: 136, SEQ ID NO: 83, SEQ ID NO: 99, SEQ ID NO: 135, SEQ ID NO: 109, or SEQ ID NO: 111 in the expression regulatory region of human DMPK gene.

(32) The ribonucleoprotein of the above-mentioned (29), wherein the guide RNA comprises the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186, or the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186 in which 1 to 3 bases are deleted, substituted, inserted, and/or added.

(33) The ribonucleoprotein of any of the above-mentioned (29) to (32), wherein the transcriptional repressor is selected from the group KRAB, MeCP2, SIN3A, HDT1, MBD2B, NIPP1, and HP1A.

(34) The ribonucleoprotein of any of the above-mentioned (29) to (33), wherein the transcriptional repressor is KRAB.

(35) The ribonucleoprotein of any of the above-mentioned (29) to (34), wherein the nuclease-deficient CRISPR effector protein is dCas9.

(36) The ribonucleoprotein of the above-mentioned (35), wherein the dCas9 is derived from Staphylococcus aureus.

(37) The ribonucleoprotein of any of (29) to (36), wherein the guide RNA comprises the base sequence set forth in SEQ ID NO: 164, SEQ ID NO: 169, SEQ ID NO: 171, or SEQ ID NO: 177, or the base sequence set forth in SEQ ID NO: 164, SEQ ID NO: 169, SEQ ID NO: 171, or SEQ ID NO: 177 in which 1 to 3 bases are deleted, substituted, inserted, and/or added,

wherein the transcriptional repressor is KRAB, and

wherein the nuclease-deficient CRISPR effector protein is dCas9 derived from Staphylococcus aureus.

(38) The ribonucleoprotein of (37), wherein the guide RNA comprises the base sequence set forth in SEQ ID NO: 171, or the base sequence set forth in SEQ ID NO: 171 in which 1 to 3 bases are deleted, substituted, inserted, and/or added.

(39) A composition or kit for suppressing an expression of human DMPK gene, comprising the following:

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene, or a polynucleotide encoding the guide RNA.

(40) The composition or kit of the above-mentioned (39), comprising the following:

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 127, SEQ ID NO: 46, SEQ ID NO: 128, SEQ ID NO: 66, SEQ ID NO: 68, SEQ ID NO: 130, SEQ ID NO: 131, SEQ ID NO: 132, SEQ ID NO: 88, SEQ ID NO: 91, SEQ ID NO: 96, SEQ ID NO: 99, SEQ ID NO: 100, SEQ ID NO: 134, SEQ ID NO: 108, SEQ ID NO: 109, SEQ ID NO: 111, SEQ ID NO: 117, or SEQ ID NO: 119 in the expression regulatory region of human DMPK gene, or a polynucleotide encoding the guide RNA.

(41) The composition or kit of the above-mentioned (39) or (40), comprising the following:

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 63, SEQ ID NO: 136, SEQ ID NO: 83, SEQ ID NO: 99, SEQ ID NO: 135, SEQ ID NO: 109, or SEQ ID NO: 111 in the expression regulatory region of human DMPK gene, or a polynucleotide encoding the guide RNA.

(42) The composition or kit of the above-mentioned (39), wherein the guide RNA comprises the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186, or the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186 in which 1 to 3 bases are deleted, substituted, inserted, and/or added.

(43) The composition or kit of the above-mentioned (39) to (42), comprising at least two different guide RNAs, or a polynucleotide encoding at least two different guide RNAs, or at least two polynucleotides encoding the guide RNAs, wherein the at least two polynucleotides are different.

(44) The composition or kit of any of the above-mentioned (39) to (43), wherein the transcriptional repressor is selected from the group KRAB, MeCP2, SIN3A, HDT1, MBD2B, NIPP1, and HP1A.

(45) The composition or kit of the above-mentioned (44), wherein the transcriptional repressor is KRAB.

(46) The composition or kit of any of the above-mentioned (39) to (45), wherein the nuclease-deficient CRISPR effector protein is dCas9.

(47) The composition or kit of the above-mentioned (46), wherein the dCas9 is derived from Staphylococcus aureus.

(48) The composition or kit of any of the above-mentioned (39) to (47),

wherein the composition or kit comprises a polynucleotide encoding the fusion protein and a polynucleotide encoding the guide RNA and

wherein the polynucleotide encoding the fusion protein further comprises a promoter sequence for the fusion protein and/or the polynucleotide encoding the guide RNA further comprises a promoter sequence for the guide RNA.

(49) The composition or kit of the above-mentioned (48), wherein the promoter sequence for the guide RNA is selected from the group U6 promoter, SNR6 promoter, SNR52 promoter, SCR1 promoter, RPR1 promoter, U3 promoter, and H1 promoter.

(50) The composition or kit of the above-mentioned (48), wherein the promoter sequence for the fusion protein is a ubiquitous promoter or a muscle specific promoter.

(51) The composition or kit of the above-mentioned (50), wherein the ubiquitous promoter is selected from the group EFS promoter, CMV promoter and CAG promoter.

(52) The composition or kit of the above-mentioned (50), wherein the muscle specific promoter is selected from the group CK8 promoter, myosin heavy chain kinase (MHCK) promoter, muscle creatine kinase (MCK) promoter, synthetic C5-12 (Syn) promoter, and Des promoter.

(53) The composition or kit of any of the above-mentioned (48) to (52), wherein the guide RNA comprises the base sequence set forth in SEQ ID NO: 164, SEQ ID NO: 169, SEQ ID NO: 171, or SEQ ID NO: 177, or the base sequence set forth in SEQ ID NO: 164, SEQ ID NO: 169, SEQ ID NO: 171, or SEQ ID NO: 177 in which 1 to 3 bases are deleted, substituted, inserted, and/or added,

wherein the transcriptional repressor is KRAB,

wherein the nuclease-deficient CRISPR effector protein is dCas9 derived from Staphylococcus aureus,

wherein the promoter sequence for the guide RNA is U6 promoter, and

wherein the promoter sequence for the fusion protein is CK8 promoter.

(54) The composition or kit of the above-mentioned (53), wherein the guide RNA comprises the base sequence set forth in SEQ ID NO: 171, or the base sequence set forth in SEQ ID NO: 171 in which 1 to 3 bases are deleted, substituted, inserted, and/or added.

(55) A method for treating or preventing myotonic dystrophy type 1, comprising a step of administering the following (e) and (f):

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene, or a polynucleotide encoding the guide RNA.

(56) The method of the above-mentioned (55), comprising a step of administering the following (e) and (f):

(e) a fusion protein of a nuclease-deficient CRISPR effector protein and a transcriptional repressor, or a polynucleotide encoding the fusion protein, and

(f) a guide. RNA targeting a continuous region of 18 to 24 nucleotides in length in a region set forth in SEQ ID NO: 127, SEQ ID NO: 46, SEQ ID NO: 128, SEQ ID NO: 66, SEQ ID NO: 68, SEQ ID NO: 130, SEQ ID NO: 131, SEQ ID NO: 132, SEQ ID NO: 88, SEQ ID NO: 91, SEQ ID NO: 96, SEQ ID NO: 99, SEQ ID NO: 100, SEQ ID NO: 134, SEQ ID NO: 108, SEQ ID NO: 109, SEQ ID NO: 111, SEQ ID NO: 117, or SEQ ID NO: 119 in the expression regulatory region of human DMPK gene, or a polynucleotide encoding the guide RNA.

(57) The method of the above-mentioned (55) or (56), comprising a step of administering the following (e) and (f):

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 63, SEQ ID NO: 136, SEQ ID NO: 83, SEQ ID NO: 99, SEQ ID NO: 135, SEQ ID NO: 109, or SEQ ID NO: 111 in the expression regulatory region of human DMPK gene, or a polynucleotide encoding the guide RNA.

(58) The method of the above-mentioned (55), wherein the guide RNA comprises the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ. ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186, or the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186 in which 1 to 3 bases are deleted, substituted, inserted, and/or added.

(59) The method of the above-mentioned (55) to (58), comprising administering at least two different guide RNAs, or a polynucleotide encoding at least two different guide RNAs, or at least two polynucleotides encoding the guide RNAs, wherein the at least two polynucleotides are different.

(60) The method of the above-mentioned (55) to (59), wherein the transcriptional repressor is selected from the group KRAB, MeCP2, SIN3A, HDT1, MBD2B, NIPP1, and HP1A.

(61) The method of the above-mentioned (60), wherein the transcriptional repressor is KRAB.

(62) The method of any of the above-mentioned (55) to (61), wherein the nuclease-deficient CRISPR effector protein is dCas9.

(63) The method of the above-mentioned (62), wherein the dCas9 is derived from Staphylococcus aureus.

(64) The method of any of the above-mentioned (55) to (63),

wherein the method comprises administering a polynucleotide encoding the fusion protein and a polynucleotide encoding the guide RNA and

wherein the polynucleotide encoding the fusion protein further comprises a promoter sequence for the fusion protein and/or the polynucleotide encoding the guide RNA further comprises a promoter sequence for the guide RNA.

(65) The method of the above-mentioned (64), wherein the promoter sequence for the guide RNA is selected from the group U6 promoter, SNR6 promoter, SNR52 promoter, SCR1 promoter, RPR1 promoter, U3 promoter, and H1 promoter.

(66) The method of the above-mentioned (64), wherein the promoter sequence for the fusion protein is a ubiquitous promoter or a muscle specific promoter.

(67) The method of the above-mentioned (66), wherein the ubiquitous promoter is selected from the group EFS promoter, CMV promoter and CAG promoter.

(68) The method of the above-mentioned (66), wherein the muscle specific promoter is selected from the group CK8 promoter, myosin heavy chain kinase (MHCK) promoter, muscle creatine kinase (MCK) promoter, synthetic C5-12 (Syn) promoter, and Des promoter.

(69) The method of any of the above-mentioned (64) to (68), wherein the guide RNA comprises the base sequence set forth in SEQ ID NO: 164, SEQ ID NO: 169, SEQ ID NO: 171, or SEQ ID NO: 177, or the base sequence set forth in SEQ ID NO: 164, SEQ ID NO: 169, SEQ ID NO: 171, or SEQ ID NO: 177 in which 1 to 3 bases are deleted, substituted, inserted, and/or added,

wherein the transcriptional repressor is KRAB,

wherein the nuclease-deficient CRISPR effector protein is dCas9 derived from Staphylococcus aureus,

wherein the promoter sequence for the guide RNA is U6 promoter, and

wherein the promoter sequence for the fusion protein is CK8 promoter.

(70) The method of the above-mentioned (69), wherein the guide RNA comprises the base sequence set forth in SEQ ID NO: 171, or the base sequence set forth in SEQ ID NO: 171 in which 1 to 3 bases are deleted, substituted, inserted, and/or added.

(71) Use of

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene, or a polynucleotide encoding the guide RNA

in the manufacture of a pharmaceutical composition for the treatment or prevention of myotonic dystrophy type 1.

(72) The use of the following (e) and (f) of the above-mentioned (71):

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 127, SEQ ID NO: 46, SEQ ID NO: 128, SEQ ID NO: 66, SEQ ID NO: 68, SEQ ID NO: 130, SEQ ID NO: 131, SEQ ID NO: 132, SEQ ID NO: 88, SEQ ID NO: 91, SEQ ID NO: 96, SEQ ID NO: 99, SEQ ID NO: 100, SEQ ID NO: 134, SEQ ID NO: 108, SEQ ID NO: 109, SEQ ID NO: 111, SEQ ID NO: 117, or SEQ ID NO: 119 in the expression regulatory region of human DMPK gene, or a polynucleotide encoding the guide RNA

in the manufacture of a pharmaceutical composition for the treatment or prevention of myotonic dystrophy type 1.

(73) The use of the following (e) and (f) of the above-mentioned (71) or (72):

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 63, SEQ ID NO: 136, SEQ ID NO: 83, SEQ ID NO: 99, SEQ ID NO: 135, SEQ ID NO: 109, or SEQ ID NO: 111 in the expression regulatory region of human DMPK gene, or a polynucleotide encoding the guide RNA

in the manufacture of a pharmaceutical composition for the treatment or prevention of myotonic dystrophy type 1.

(74) The use of the above-mentioned (71), wherein the guide RNA comprises the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186, or the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186 in which 1 to 3 bases are deleted, substituted, inserted, and/or added.

(75) The use of the above-mentioned (71) to (74), comprising use of at least two different guide RNAs, or a polynucleotide encoding at least two different guide RNAs, or at least two polynucleotides encoding the guide RNAs, wherein the at least two polynucleotides are different.

(76) The use of the above-mentioned (71) to (75), wherein the transcriptional repressor is selected from the group KRAB, MeCP2, SIN3A, HDT1, MBD2B, NIPP1, and HP1A.

(77) The use of the above-mentioned (76), wherein the transcriptional repressor is KRAB.

(78) The use of any of the above-mentioned (71) to (77), wherein the nuclease-deficient CRISPR effector protein is dCas9.

(79) The use of the above-mentioned (78), wherein the dCas9 is derived from Staphylococcus aureus.

(80) The use of any of the above-mentioned (71) to (79),

wherein the use comprises use of a polynucleotide encoding the fusion protein and use of a polynucleotide encoding the guide RNA and

wherein the polynucleotide encoding the fusion protein further comprises a promoter sequence for the fusion protein and/or the polynucleotide encoding the guide RNA further comprises a promoter sequence for the guide RNA.

(81) The use of the above-mentioned (80), wherein the promoter sequence for the guide RNA is selected from the group U6 promoter, SNR6 promoter, SNR52 promoter, SCR1 promoter, RPR1 promoter, U3 promoter, and H1 promoter.

(82) The use of the above-mentioned (80), wherein the promoter sequence for the fusion protein is a ubiquitous promoter or a muscle specific promoter.

(83) The use of the above-mentioned (82), wherein the ubiquitous promoter is selected from the group EFS promoter, CMV promoter and CAG promoter.

(84) The use of the above-mentioned (82), wherein the muscle specific promoter is selected from the group CK8 promoter, myosin heavy chain kinase (MHCK) promoter, muscle creatine kinase (MCK) promoter, synthetic C5-12 (Syn) promoter, and Des promoter.

(85) The use of the above-mentioned (80) to (84), wherein the guide RNA comprises the base sequence set forth in SEQ ID NO: 164, SEQ ID NO: 169, SEQ ID NO: 171, or SEQ ID NO: 177, or the base sequence set forth in SEQ ID NO: 164, SEQ ID NO: 169, SEQ ID NO: 171, or SEQ ID NO: 177 in which 1 to 3 bases are deleted, substituted, inserted, and/or added,

wherein the transcriptional repressor is KRAB,

wherein the nuclease-deficient CRISPR effector protein is dCas9 derived from Staphylococcus aureus,

wherein the promoter sequence for the guide RNA is U6 promoter, and

wherein the promoter sequence for the fusion protein is CK8 promoter.

(86) The use of the above-mentioned (85), wherein the guide RNA comprises the base sequence set forth in SEQ ID NO: 171, or the base sequence set forth in SEQ ID NO: 171 in which 1 to 3 bases are deleted, substituted, inserted, and/or added.

### Effect of the Invention

According to the present invention, the expression of human DMPK gene can be suppressed and, consequently, the present invention is expected to be able to treat and/or prevent DM1.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the positions of the targeting sequences set forth in SEQ ID NOs: 4 to 126, in which the black box shows the position of the targeting sequence that showed not less than 50% reduction in human DMPK gene expression.

FIG. 2 shows the results of the expression suppressive action on the human DMPK gene evaluated using dSaCas9-KRAB and sgRNA comprising crRNA encoded by the targeting sequence set forth in SEQ ID NOs: 4 to 126, respectively. The horizontal axis shows sgRNA comprising crRNA encoded by each targeting sequence, the vertical axis shows the ratio of expression level of DMPK gene when using each sgRNA to the expression level (100%) of DMPK gene when using control sgRNA, and the error bar shows standard deviation.

FIG. 3 shows the relationship between the positions of the targeting sequences set forth in SEQ ID NOs: 4 to 126 and the expression level of the human DMPK gene when the expression of the human DMPK gene was controlled using dSaCas9-KRAB and sgRNA comprising crRNA encoded by the targeting sequences, respectively.

FIG. 4 shows DMPK downregulation in human muscular cells.

FIG. 5 shows that AAV9-695 suppressed DMPK expression in DMSXL mice (A; Tibialis Anterior, B; Heart, C; Liver).

FIG. 6 shows that AAV9-245 suppressed DMPK expression in DMSXL mice (A; Tibialis Anterior, B; Heart, C; Liver).

FIG. 7 shows that AAV9-257 suppressed DMPK expression in DMSXL mice (A; Tibialis Anterior, B; Heart, C; Liver).

FIG. 8 shows that AAV9-695 improved RNA foci formation in DMSXL mice.

FIG. 9 shows suppression of DMPK gene expression in hDMPK sgRNA-expressing iDM cells.

FIG. 10 shows improvement of RNA foci formation in hDMPK sgRNA-expressing iDM cells (A; Typical images of iDM-695 cells and iDM-Ctrl cells, B; The ratios of RNA foci positive nuclei in each cell).

FIG. 11 shows improvement of splicing defects in hDMPK sgRNA-expressing iDM cells (A; Gel images and exon patterns of the genes, B; The ratios of normally spliced products).

### DETAILED DESCRIPTION OF THE PREFERRED EMBODIMENTS

### 1. Polynucleotide

The present invention provides a polynucleotide comprising the following base sequences (hereinafter sometimes to be also referred to as “the polynucleotide of the present invention”):

(a) a base sequence encoding a fusion protein of a nuclease-deficient CRISPR effector protein and a transcriptional repressor, and

(b) a base sequence encoding a guide. RNA targeting a continuous region of 18 to 24 nucleotides (i.e., 18 to 24 contiguous nucleotides) in length in a region set forth in SEQ. ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene.

The polynucleotide of the present invention is introduced into a desired cell and transcribed to produce a fusion protein of a nuclease-deficient CRISPR effector protein and a transcriptional repressor, and a guide RNA targeting a particular region of the expression regulatory region of the human DMPK gene. These fusion protein and guide RNA form a complex (hereinafter the complex is sometimes referred to as “ribonucleoprotein; RNP”) and cooperatively act on the aforementioned particular region, thus repressing transcription of the human DMPK gene. In one embodiment of the present invention, the expression of the human DMPK gene can be suppressed by, for example, not less than about 40%, not less than about 50%, not less than about 60%, not less than about 70%, not less than about 75%, not less than about 80%, not less than about 85%, not less than about 90%, not less than about 95%, or about 100%.

### (1) Definition

In the present specification, “the expression regulatory region of human DMPK gene” means any region in which the expression of human DMPK gene can be repressed by binding RNP to that region. That is, the expression regulatory region of human DMPK gene may exist in any region such as the promoter region, enhancer region, intron, exon of the human DMPK gene, and neighboring genes of human DMPK gene (e.g., human DMWD (DM1 locus, WD repeat containing) gene), as long as the expression of the human DMPK gene is repressed by the binding of RNP. In the present specification, when the expression regulatory region is shown by the particular sequence, the expression regulatory region includes both the sense strand sequence and the antisense strand sequence conceptually.

In the present invention, a fusion protein of a nuclease-deficient CRISPR effector protein and a transcriptional repressor is recruited by a guide RNA into a particular region in the expression regulatory region of the human DMPK gene. In the present specification, the “guide RNA targeting . . . ” means a “guide RNA recruiting a fusion protein into . . . ”.

In the present specification, the “guide RNA (to be also referred to as ‘gRNA’)” is an RNA comprising a genome specific CRISPR-RNA (to be referred to as “crRNA”). crRNA is an RNA that binds to a complementary sequence of a targeting sequence (described later). When Cpf1 is used as the CRISPR effector protein, the “guide RNA” refers to an RNA comprising an RNA consisting of crRNA and a specific sequence attached to its 5′-terminal (for example, an RNA sequence set forth in SEQ ID NO: 138 in the case of FnCpf1). When Cas9 is used as the CRISPR effector protein, the “guide RNA” refers to a chimera RNA (to be referred to as “single guide RNA(sgRNA)”) comprising crRNA and trans-activating crRNA attached to its 3′-terminal (to be referred to as “tracrRNA”) (see, for example, Zhang F. et al., Hum Mol Genet. 2014 Sep. 15; 23(R1):R40-6 and Zetsche B. et al., Cell. 2015 Oct. 22; 163(3): 759-71, which are incorporated herein by reference in their entireties).

In the present specification, a sequence complementary to the sequence to which crRNA binds in the expression regulatory region of the human DMPK gene is referred to as a “targeting sequence”. That is, in the present specification, the “targeting sequence” is a DNA sequence present in the expression regulatory region of the human DMPK gene and adjacent to PAM (protospacer adjacent motif). PAM is adjacent to the 5′-side of the targeting sequence when Cpf1 is used as the CRISPR effector protein. PAM is adjacent to the 3′-side of the targeting sequence when Cas9 is used as the CRISPR effector protein. The targeting sequence may be present on either the sense strand sequence side or the antisense strand sequence side of the expression regulatory region of the human DMPK gene (see, for example, the aforementioned Zhang F. et al., Hum Mol Genet. 2014 Sep. 15; 23(R1):R40-6 and Zetsche B. et al., Cell. 2015 Oct. 22; 163(3): 759-71, which are incorporated herein by reference in their entireties).

### (2) Nuclease-Deficient CRISPR Effector Protein

In the present invention, using a nuclease-deficient CRISPR effector protein, a transcriptional repressor fused thereto is recruited to the expression regulatory region of the human DMPK gene. The nuclease-deficient CRISPR effector protein (hereinafter sometimes to be simply referred to as “CRISPR effector protein”) to be used in the present invention is not particularly limited as long as it forms a complex with gRNA and is recruited to the expression regulatory region of the human DMPK gene. For example, nuclease-deficient Cas9 (hereinafter sometimes to be also referred to as “dCas9”) or nuclease-deficient Cpf1 (hereinafter sometimes to be also referred to as “dCpf1”) can be included.

Examples of the above-mentioned dCas9 include, but are not limited to, a nuclease-deficient variant of Streptococcus pyogenes-derived Cas9 (SpCas9; PAM sequence: NGG (N is A, G, T or C. hereinafter the same)), Streptococcus thermophilus-derived Cas9 (St1Cas9; PAM sequence: NNAGAAW (W is A or T. hereinafter the same), St3Cas9; PAM sequence: NGGNG), Neisseria meningitidis-derived Cas9 (NmCas9; PAM sequence: NNNNGATT), or Staphylococcus aureus-derived Cas9 (SaCas9; PAM sequence: NNGRRT (R is A or G. hereinafter the same)) and the like (see, for example, Nishimasu et al., Cell. 2014 Feb. 27; 156(5): 935-49, Esvelt K M et al., Nat Methods. 2013 November; 10(11):1116-21, Zhang Y. Mol Cell. 2015 Oct. 15; 60(2):242-55, and Friedland A E et al., Genome Biol. 2015 Nov. 24; 16:257, which are incorporated herein by reference in their entireties). For example, in the case of SpCas9, a double mutant in which the Asp residue at the 10th position is converted to Ala residue and the His residue at the 840th position is converted to Ala residue (sometimes referred to as “dSpCas9”) can be used (see, for example, the aforementioned Nishimasu et al., Cell. 2014, which is incorporated herein by reference in their entireties). Alternatively, in the case of SaCas9, a double mutant in which the Asp residue at the 10th position is converted to Ala residue and the Asn residue at the 580th position is converted to Ala residue (SEQ ID NO: 139), or a double mutant in which the Asp residue at the 10th position is converted to Ala residue and the His residue at the 557th position is converted to Ala residue (SEQ ID NO: 140) (hereinafter any of these double mutants is sometimes to be referred to as “dSaCas9”) can be used (see, for example, the aforementioned Friedland A E et al., Genome Biol. 2015, which are incorporated herein by reference in their entireties).

In addition, in one embodiment of the present invention, as dCas9, a variant obtained by modifying a part of the amino acid sequence of the aforementioned dCas9, which forms a complex with gRNA and is recruited to the expression regulatory region of the human DMPK gene, may also be used. Examples of such variants include a truncated variant with a partly deleted amino acid sequence. In one embodiment of the present invention, the variant described in WO2019/235627 and WO2020/085441, which are incorporated herein by reference in their entireties, can be used as dCas9. Specifically, dSaCas9 obtained by deleting the 721st to 745th amino acids from dSaCas9 that is a double mutant in which the Asp residue at the 10th position is converted to Ala residue and the Asn residue at the 580th position is converted to Ala residue (SEQ ID NO: 141), or dSaCas9 in which the deleted part is substituted by a peptide linker (e.g., one in which the deleted part is substituted by GGSGGS linker (SEQ ID NO: 142) is set forth in SEQ ID NO: 143) (hereinafter any of these double mutants is sometimes to be referred to as “dSaCas9[-25]”), or dSaCas9 obtained by deleting the 482nd to 648th amino acids of dSaCas9 that is the aforementioned double mutant (SEQ ID NO: 144), or dSaCas9 in which the deleted part is substituted by a peptide linker (one in which the deleted part is substituted by GGSGGS linker is set forth in SEQ ID NO: 145) may also be used.

Examples of the above-mentioned dCpf1 include, but are not limited to, a nuclease-deficient variant of Francisella novicida-derived Cpf1 (FnCpf1; PAM sequence: TTN), Acidaminococcus sp.-derived Cpf1 (AsCpf1; PAM sequence: TTTN), or Lachnospiraceae bacterium-derived Cpf1 (LbCpf1; PAM sequence: TTTA, TCTA, TCCA, or CCCA) and the like (see, for example, Zetsche B. et al., Cell. 2015 Oct. 22; 163(3):759-71, Yamano T et al., Cell. 2016 May 5; 165(4):949-62, and Yamano T et al., Mol Cell. 2017 Aug. 17; 67(4):633-45, which are incorporated herein by reference in their entireties). For example, in the case of FnCpf1, a double mutant in which the Asp residue at the 917th position is converted to Ala residue and the Glu residue at the 1006th position is converted to Ala residue can be used (see, for example, the aforementioned Zetsche B et al., Cell. 2015, which is incorporated herein by reference in its entirety). In one embodiment of the present invention, as dCpf1, a variant obtained by modifying a part of the amino acid sequence of the aforementioned dCpf1, which forms a complex with gRNA and is recruited to the expression regulatory region of the human DMPK gene, may also be used.

In one embodiment of the present invention, dCas9 is used as the nuclease-deficient CRISPR effector protein. In one embodiment, the dCas9 is dSaCas9, and, in a particular embodiment, dSaCas9 is dSaCas9[-25].

A polynucleotide comprising a base sequence encoding a nuclease-deficient CRISPR effector protein can be cloned by, for example, synthesizing an oligoDNA primer covering a region encoding a desired part of the protein based on the cDNA sequence information thereof, and amplifying the polynucleotide by PCR method using total RNA or mRNA fraction prepared from the cells producing the protein as a template. In addition, a polynucleotide comprising a base sequence encoding a nuclease-deficient CRISPR effector protein can be obtained by introducing a mutation into a nucleotide sequence encoding a cloned CRISPR effector protein by a known site-directed mutagenesis method to convert the amino acid residues (e.g., Asp residue at the 10th position, His residue at the 557^{th} position, and Asn residue at the 580th position in the case of SaCas9; Asp residue at the 917^{th} position and Glu residue at the 1006^{th} position in the case of FnCpf1, and the like can be included, but are not limited to these) at a site important for nuclease activity to other amino acids.

Alternatively, a polynucleotide comprising a base sequence encoding nuclease-deficient CRISPR effector protein can be obtained by chemical synthesis or a combination of chemical synthesis and PCR method or Gibson Assembly method, based on the cDNA sequence information thereof, and can also be further constructed as a base sequence that underwent codon optimization to be codons suitable for expression in human.

### (3) Transcriptional Repressor

In the present invention, human DMPK gene expression is repressed by the action of the transcriptional repressor fused with the nuclease-deficient CRISPR effector protein. In the present specification, the “transcriptional repressor” means a protein having the ability to repress gene transcription of human DMPK gene or a peptide fragment retaining the function thereof. The transcriptional repressor to be used in the present invention is not particularly limited as long as it can repress expression of human DMPK gene. It includes, for example, Kruppel-associated box (KRAB), MBD2B, v-ErbA, SID (including chain state of SID (SID4X)), MBD2, MBD3, DNMT family (e.g., DNMT1, DNMT3A, DNMT3B), Rb, MeCP2, ROM2, LSD1, AtHD2A, SET1, HDAC11, SETD8, EZH2, SUV39H1, PHF19, SALI, NUE, SUVR4, KYP, DIM5, HDAC8, SIRT3, SIRT6, MESOL04, SET8, HST2, COBB, SET-TAF1B, NCOR, SIN3A, HDT1, NIPP1, HP1A, ERF repressor domain (ERD), and variants thereof having transcriptional repression ability, fusions thereof and the like. In one embodiment of the present invention, KRAB is used as the transcriptional repressor.

A polynucleotide comprising a base sequence encoding a transcriptional repressor can be constructed by chemical synthesis or a combination of chemical synthesis and PCR method or Gibson Assembly method. Furthermore, a polynucleotide comprising a base sequence encoding a transcriptional repressor can also be constructed as a codon-optimized DNA sequence to be codons suitable for expression in human.

A polynucleotide comprising a base sequence encoding a fusion protein of a transcriptional repressor and a nuclease-deficient CRISPR effector protein can be prepared by ligating a base sequence encoding the CRISPR effector protein to a base sequence encoding the transcriptional repressor directly or after adding a base sequence encoding a linker, NLS (nuclear localization signal)(for example, a base sequence set forth in SEQ ID NO: 189 or SEQ ID NO: 191), a tag and/or others. In the present invention, the transcriptional repressor may be fused with either N-terminal or C-terminal of the nuclease-deficient CRISPR effector protein. As the linker, a linker with an amino acid number of about 2 to 50 can be used, and specific examples thereof include, but are not limited to, a G-S-G-S linker in which glycine (G) and serine (S) are alternately linked and the like. In one embodiment of the present invention, as the polynucleotide comprising a base sequence encoding a fusion protein of a nuclease-deficient CRISPR effector protein and a transcriptional repressor, the base sequence set forth in SEQ ID NO: 151, which encodes SV40 NLS, dSaCas9, NLS and KRAB as a fused protein, can be used. (4) Guide RNA

In the present invention, a fusion protein of nuclease-deficient CRISPR effector protein and transcriptional repressor can be recruited to the expression regulatory region of the human DMPK gene by guide RNA. As described in the aforementioned “(1) Definition”, guide RNA comprises crRNA, and the crRNA binds to a complementary sequence of the targeting sequence. crRNA may not be completely complementary to the complementary sequence of the targeting sequence as long as the guide RNA can recruit the fusion protein to the target region, and may be a sequence in which at least 1 to 3 bases are deleted, substituted, inserted and/or added.

When dCas9 is used as the nuclease-deficient CRISPR effector protein, for example, the targeting sequence can be determined using a published gRNA design web site (CRISPR Design Tool, CRISPR direct etc.). To be specific, from the sequence of the target gene (i.e., human DMPK gene) and neighboring gene thereof, candidate targeting sequences of about 20 nucleotides in length for which PAM (e.g., NNGRRT in the case of SaCas9) is adjacent to the 3′-side thereof are listed, and one having a small number of off-target sites in human genome among these candidate targeting sequences can be used as the targeting sequence. The base length of the targeting sequence is 18 to 24 nucleotides in length, preferably 18 to 23 nucleotides in length, more preferably 18 to 22 nucleotides in length. As a primary screening for the prediction of the off-target site number, a number of bioinformatic tools are known and publicly available, and can be used to predict the targeting sequence with the lowest off-target effect. Examples thereof include bioinformatics tools such as Benchling (https://benchling.com), and COSMID (CRISPR Off-target Sites with Mismatches, Insertions and Deletions) (Available on https://crispr.bme.gatech.edu on the internet). Using these, the similarity to the base sequence targeted by gRNA can be summarized. When the gRNA design software to be used does not have a function to search for off-target site of the target genome, for example, the off-target site can be searched for by subjecting the target genome to Blast search with respect to 8 to 12 nucleotides on the 3′-side of the candidate targeting sequence (seed sequence with high discrimination ability of targeted nucleotide sequence).

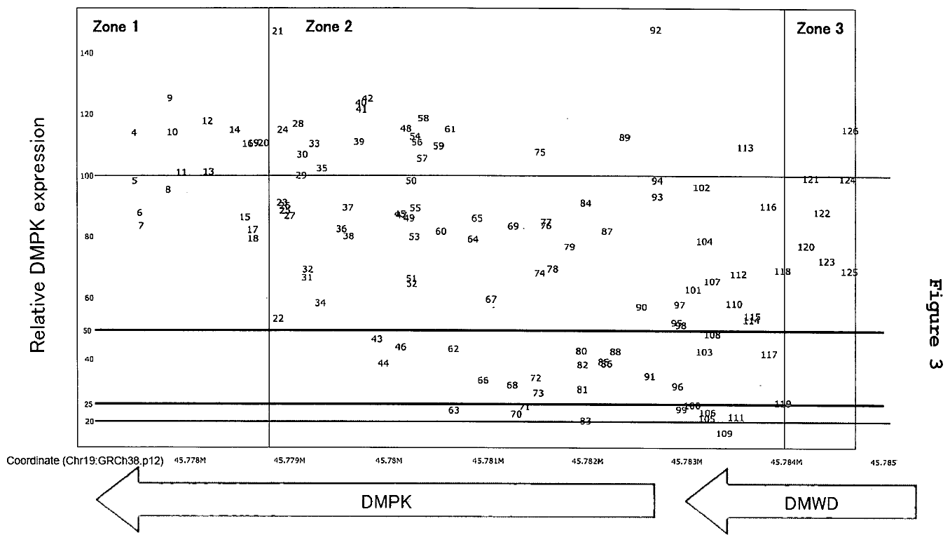

In one embodiment of the present invention, in the region existing in the GRCh38.p12 position of human chromosome 19 (Chr19), region near the transcription start point of the DMPK gene: 45,777,342-45,784,715 can be the expression regulatory region of human DMPK gene. As shown in the Examples, the present inventors have found that the expression of human DMPK gene can be regulated by targeting the region 45,778,884-45,783,985 (Zone 2 in FIG. 3), in the above-mentioned regions. In one embodiment of the present invention, therefore, the targeting sequence may be a base sequence of continuous 18 to 24 nucleotides in length, preferably 18 to 23 nucleotides in length, more preferably 18 to 22 nucleotides in length, in the following region: 45,778,884-45,783,985 in the region existing in the GRCh38.p12 position of human chromosome 19 (Chr19).

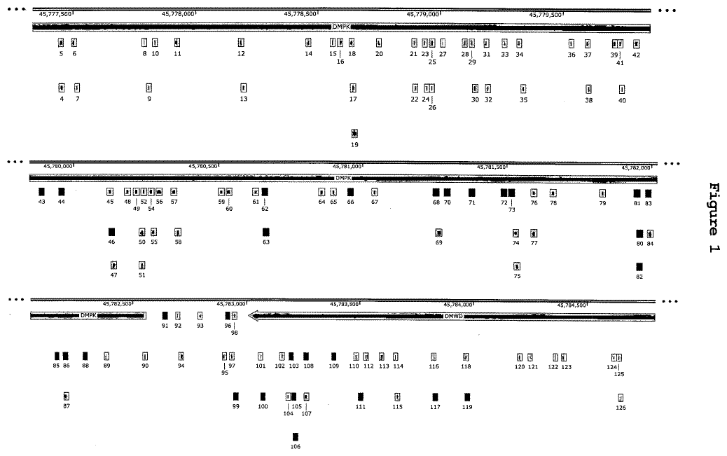

Furthermore, the present inventors have found that the region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 existing in the above-mentioned region 45,778,884-45,783,985 is preferable as a region for designing the targeting sequence for repressing the expression of DMPK gene. In one embodiment of the present invention, therefore, the targeting sequence may be a base sequence of continuous 18 to 24 nucleotides in length, preferably 18 to 23 nucleotides in length, more preferably 18 to 22 nucleotides in length, in these regions. The position of each sequence in the expression regulatory region of human DMPK gene is as described in Table 1 and FIG. 1.

In one embodiment of the present invention, the targeting sequence may be a base sequence of continuous 18 to 24 nucleotides in length, preferably 18 to 23 nucleotides in length, more preferably 18 to 22 nucleotides in length, in the region set forth in SEQ ID NO: 127, SEQ ID NO: 46, SEQ ID NO: 128, SEQ ID NO: 66, SEQ ID NO: 68, SEQ ID NO: 130, SEQ ID NO: 131, SEQ ID NO: 132, SEQ ID NO: 88, SEQ ID NO: 91, SEQ ID NO: 96, SEQ ID NO: 99, SEQ ID NO: 100, SEQ ID NO: 134, SEQ ID NO: 108, SEQ ID NO: 109, SEQ ID NO: 111, SEQ ID NO: 117, or SEQ ID NO: 119, existing in the above-mentioned regions 45,778,884-45,783,985, which is considered to show not less than 50% reduction in human DMPK gene expression. The position of each sequence in the expression regulatory region of human DMPK gene is as described in Table 1 and FIG. 1.

In another embodiment of the present invention, the targeting sequence may be a base sequence of continuous 18 to 24 nucleotides in length, preferably 18 to 23 nucleotides in length, more preferably 18 to 22 nucleotides in length, in the region set forth in SEQ ID NO: 63, SEQ ID NO: 136, SEQ ID NO: 83, SEQ ID NO: 99, SEQ ID NO: 135, SEQ ID NO: 109, or SEQ ID NO: 111, existing in the above-mentioned regions 45,778,884-45,783,985, which is considered to show not less than 75% reduction human DMPK gene expression. The position of each sequence in the expression regulatory region of human DMPK gene is as described in Table 1 and FIG. 1.

In still another embodiment of the present invention, the targeting sequence may be a base sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117, or SEQ ID NO: 119. The base sequences set forth in SEQ ID NOs: 43 and 44 are targeting sequences comprised in a region set forth in SEQ ID NO: 127. The base sequences set forth in SEQ ID NOs: 62 and 63 are targeting sequences comprised in a region set forth in SEQ ID NO: 128. The base sequences set forth in SEQ ID NOs: 66 to 68 are targeting sequences comprised in a region set forth in SEQ ID NO: 129. The base sequences set forth in SEQ ID NOs: 70 to 73 are targeting sequences comprised in a region set forth in SEQ ID NO: 130. The base sequences set forth in SEQ ID NOs: 80 to 83 are targeting sequences comprised in a region set forth in SEQ ID NO: 131. The base sequences set forth in SEQ ID NOs: 85 and 86 are targeting sequences comprised in a region set forth in SEQ ID NO: 132. The base sequences set forth in SEQ ID NOs: 95 to 100 are targeting sequences comprised in a region set forth in SEQ ID NO: 133. The base sequences set forth in SEQ ID NOs: 103, 105 and 106 are targeting sequences comprised in a region set forth in SEQ ID NO: 134. The base sequences set forth in SEQ ID NOs: 105 and 106 are targeting sequences comprised in a region set forth in SEQ ID NO: 135. The base sequences set forth in SEQ ID NOs: 70 and 71 are targeting sequences comprised in a region set forth in SEQ ID NO: 136. The base sequences set forth in SEQ ID NOs: 103 to 112 are targeting sequences comprised in a region set forth in SEQ ID NO: 137. The position of each sequence in the expression regulatory region of human DMPK gene is as described in Table 1 and FIG. 1.

In one embodiment of the present invention, a base sequence encoding crRNA may be the same base sequence as the targeting sequence. For example, when the targeting sequence set forth in SEQ ID NO: 5 (CCCAGTCGAGGCCAAAGAAGA) is introduced into the cell as a base sequence encoding crRNA, crRNA transcribed from the sequence is CCCAGUCGAGGCCAAAGAAGA (SEQ ID NO: 146) and is bound to TCTTCTTTGGCCTCGACTGGG (SEQ ID NO: 147), which is a sequence complementary to the base sequence set forth in SEQ ID NO: 5 and is present in the expression regulatory region of the human DMPK gene. In another embodiment, a base sequence which is a targeting sequence in which at least 1 to 3 bases are deleted, substituted, inserted and/or added can be used as the base sequence encoding crRNA as long as guide RNA can recruit a fusion protein to the target region. Therefore, in one embodiment of the present invention, as a base sequence encoding crRNA, the base sequence set forth in SEQ ID NO: 43, SEQ ID NO: 44, SEQ ID NO: 46, SEQ ID NO: 62, SEQ ID. NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117 or SEQ ID NO: 119, or the base sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117 or SEQ ID NO: 119 in which 1 to 3 bases are deleted, substituted, inserted and/or added can be used. In another embodiment of the present invention, as a base sequence encoding crRNA, the base sequence set forth in SEQ ID NO: 63, SEQ ID NO: 70, SEQ ID NO: 71, SEQ ID NO: 83, SEQ ID NO: 99, SEQ ID NO: 105, SEQ ID NO: 106, SEQ ID NO: 109, or SEQ ID NO: 111, or the base sequence set forth in SEQ ID NO: 63, SEQ ID NO: 70, SEQ ID NO: 71, SEQ ID NO: 83, SEQ ID NO: 99, SEQ ID NO: 105, SEQ ID NO: 106, SEQ ID NO: 109, or SEQ ID NO: 111 in which 1 to 3 bases are deleted, substituted, inserted and/or added can be used. In still another embodiment of the present invention, as a base sequence encoding crRNA, the base sequence set forth in SEQ ID NO: 70, SEQ ID NO: 81, SEQ ID NO: 83, or SEQ ID NO: 99, or the base sequence set forth in SEQ ID NO: 70, SEQ ID NO: 81, SEQ ID NO: 83, or SEQ ID NO: 99 in which 1 to 3 bases are deleted, substituted, inserted and/or added can be used. In one embodiment of the present invention, as a base sequence encoding crRNA, the base sequence set forth in SEQ ID NO: 83, or the base sequence set forth in SEQ ID NO: 83 in which 1 to 3 bases are deleted, substituted, inserted and/or added can be used.

In one embodiment of the present invention, the base sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117 or SEQ ID NO: 119, or the base sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117 or SEQ ID NO: 119 in which 1 to 3 bases are deleted, substituted, inserted and/or added can be used as the base sequence encoding crRNA to produce gRNA comprising crRNA set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186, or crRNA set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186 in which 1 to 3 bases are deleted, substituted, inserted, and/or added, respectively. In another embodiment of the present invention, the gRNA can comprise the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186, or the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186 in which 1 to 3 bases are deleted, substituted, inserted, and/or added. In one embodiment of the present invention, the gRNA can comprise the base sequence set forth in SEQ ID NO: 161, SEQ ID NO: 164, SEQ ID NO: 165, SEQ ID NO: 171, SEQ ID NO: 177, SEQ ID NO: 180, SEQ ID NO: 181, SEQ ID NO: 183, or SEQ ID NO: 184, or the base sequence set forth in SEQ ID NO: 161, SEQ ID NO: 164, SEQ ID NO: 165, SEQ ID NO: 171, SEQ ID NO: 177, SEQ ID NO: 180, SEQ ID NO: 181, SEQ ID NO: 183, or SEQ ID NO: 184 in which 1 to 3 bases are deleted, substituted, inserted, and/or added. In another embodiment of the present invention, the gRNA can comprise the base sequence set forth in SEQ ID NO: 164, SEQ ID NO: 169, SEQ ID NO: 171, or SEQ ID NO: 177, or the base sequence set forth in SEQ ID NO: 164, SEQ ID NO: 169, SEQ ID NO: 171, or SEQ ID NO: 177 in which 1 to 3 bases are deleted, substituted, inserted, and/or added. In still another embodiment of the present invention, the gRNA can comprise the base sequence set forth in SEQ ID NO: 171, or the base sequence set forth in SEQ ID NO: 171 in which 1 to 3 bases are deleted, substituted, inserted, and/or added.

When dCpf1 is used as the nuclease-deficient CRISPR effector protein, a base sequence encoding gRNA can be designed as a DNA sequence encoding crRNA with particular RNA attached to the 5′-terminal. Such RNA attached to the 5′-terminal of crRNA and a DNA sequence encoding said RNA can be appropriately selected by those of ordinary skill in the art according to the dCpf1 to be used. For example, when dFnCpf1 is used, a base sequence in which SEQ ID NO: 148; AATTTCTACTGTTGTAGAT is attached to the 5′-side of the targeting sequence can be used as a base sequence encoding gRNA (when transcribed to RNA, the sequences of the underlined parts form base pairs to form a stem-loop structure). The sequence to be added to the 5′-terminal may be a sequence generally used for various Cpf1 proteins in which at least 1 to 6 bases are deleted, substituted, inserted and/or added, as long as gRNA can recruit a fusion protein to the expression regulatory region after transcription.

When dCas9 is used as the nuclease-deficient CRISPR effector protein, a base sequence encoding gRNA can be designed as a DNA sequence in which a DNA sequence encoding known tracrRNA is linked to the 3′-terminal of a DNA sequence encoding crRNA. Such tracrRNA and a DNA sequence encoding the tracrRNA can be appropriately selected by those of ordinary skill in the art according to the dCas9 to be used. For example, when dSaCas9 is used, the base sequence set forth in SEQ ID NO: 149 is used as the DNA sequence encoding tracrRNA. The DNA sequence encoding tracrRNA may be a base sequence encoding tracrRNA generally used for various Cas9 proteins in which at least 1 to 6 bases are deleted, substituted, inserted and/or added, as long as gRNA can recruit a fusion protein to the expression regulatory region after transcription.

A polynucleotide comprising a base sequence encoding gRNA designed in this way can be chemically synthesized using a known DNA synthesis method.

In another embodiment of the present invention, the polynucleotide of the present invention may comprise at least two different base sequences respectively encoding a gRNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene. For example, the polynucleotide can comprise at least two different base sequences respectively encoding a guide RNA, wherein the at least two different base sequences are selected from a base sequence comprising a sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ. ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117 or SEQ ID NO: 119 or a base sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117 or SEQ ID NO: 119 in which 1 to 3 bases are deleted, substituted, inserted, and/or added. In one embodiment of the present invention, the polynucleotide can comprise at least two different base sequences respectively encoding a guide RNA, wherein the at least two different base sequences are selected from a base sequence comprising the sequence set forth in SEQ ID NO: 63, SEQ ID NO: 70, SEQ ID NO: 71, SEQ ID NO: 83, SEQ ID NO: 99, SEQ ID NO: 105, SEQ ID NO: 106, SEQ ID NO: 109, or SEQ ID NO: 111 or a base sequence set forth in SEQ ID NO: 63, SEQ ID NO: 70, SEQ ID NO: 71, SEQ ID NO: 83, SEQ ID NO: 99, SEQ ID NO: 105, SEQ ID NO: 106, SEQ ID NO: 109, or SEQ ID NO: 111 in which 1 to 3 bases are deleted, substituted, inserted, and/or added. In one embodiment of the present invention, the polynucleotide can comprise at least two different base sequences respectively encoding a guide RNA, wherein the at least two different base sequences are selected from a base sequence comprising the sequence set forth in SEQ ID NO: 70, SEQ ID NO: 81, SEQ ID NO: 83 or SEQ ID NO: 99 or a base sequence set forth in SEQ ID NO: 70, SEQ ID NO: 81, SEQ ID NO: 83 or SEQ ID NO: 99 in which 1 to 3 bases are deleted, substituted, inserted, and/or added.

### (5) Promoter Sequence

In one embodiment of the present invention, a promoter sequence may be operably linked to the upstream of each of a base sequence encoding fusion protein of nuclease-deficient CRISPR effector protein and transcriptional repressor and/or a base sequence encoding gRNA. The promoter to be possibly linked is not particularly limited as long as it shows a promoter activity in the target cell. Examples of the promoter sequence possibly linked to the upstream of the base sequence encoding gRNA include, but are not limited to, U6 promoter, SNR6 promoter, SNR52 promoter, SCR1 promoter, RPR1 promoter, U3 promoter, H1 promoter, and tRNA promoter, which are pol III promoters, and the like. In one embodiment of the present invention, U6 promoter can be used as the promoter sequence for the base sequence encoding the guide RNA. In one embodiment of the present invention, when a polynucleotide comprises two or more base sequences respectively encoding a guide RNA, a single promoter sequence may be operably linked to the upstream of the two or more base sequences. In another embodiment, when a polynucleotide comprises two or more base sequences respectively encoding a guide RNA, a promoter sequence may be operably linked to the upstream of each of the two or more base sequences, wherein the promoter sequence operably linked to each base sequence may be the same or different.

As the aforementioned promoter sequence possibly linked to the upstream of the base sequence encoding fusion protein, a ubiquitous promoter or muscle-specific promoter may be used. Examples of the ubiquitous promoter include, but are not limited to, EF-1α promoter, EFS promoter, CMV (cytomegalovirus) promoter, hTERT promoter, SRα promoter, SV40 promoter, LTR promoter, CAG promoter, RSV (Rous sarcoma virus) promoter, and the like. In one embodiment of the present invention, EFS promoter, CMV promoter or CAG promoter can be used as the ubiquitous promoter. Examples of the muscle specific promoter include, but are not limited to, CK8 promoter, CK6 promoter, CK1 promoter, CK7 promoter, CK9 promoter, cardiac muscle troponin C promoter, α-actin promoter, myosin heavy chain kinase (MHCK) promoter (e.g., MHCK7 promoter etc.), MHC promoter, myosin light chain 2A promoter, dystrophin promoter, muscle creatine kinase (MCK) promoter, dMCK promoter, tMCK promoter, enh348 MCK promoter, synthetic C5-12 (Syn) promoter, Myf5 promoter, MLC1/3f promoter, MLC-2 promoter, MYOD promoter, Myog promoter, Pax7 promoter, Des promoter, cTnC promoter and the like (for the detail of the muscle specific promoter, see, US2011/0212529A1, McCarthy J J et al., Skeletal Muscle. 2012 May; 2(1):8, Wang B. et al., Gene Ther. 2008 November; 15(22):1489-99, and the like, which are incorporated herein by reference in their entireties). In one embodiment of the present invention, CK8 promoter, myosin heavy chain kinase (MHCK) promoter, muscle creatine kinase (MCK) promoter, synthetic C5-12 (Syn) promoter, or Des promoter can be used as the muscle-specific promoter. In one embodiment of the present invention, CK8 promoter can be used as the muscle specific promoter. The aforementioned promoter may have any modification and/or alteration as long as it has promoter activity in the target cell.

In one embodiment of the present invention, U6 is used as a promoter for a base sequence encoding the guide RNA, and CK8 promoter can be used as the promoter sequence for the base sequence encoding the fusion protein. Specifically, as for the U6 promoter, the following base sequences can be used; (i) the base sequence set forth in SEQ ID NO: 155, (ii) a base sequence set forth in SEQ ID NO: 155 wherein 1 or several (e.g., 2, 3, 4, 5 or more) bases are deleted, substituted, inserted and/or added with a promoter activity in the target cell, or (iii) a base sequence not less than 90% (e.g., 90%, 91%, 92%, 93%, 94%, 95%, 96%, 97%, 98%, 99% or above) identical with the base sequence set forth in SEQ ID NO: 155 showing a promoter activity in the target cell. As for the CK8 promoter, the following base sequences can be used; (i) the base sequence set forth in SEQ ID NO: 187, (ii) a base sequence set forth in SEQ ID NO: 187 wherein 1 or several (e.g., 2, 3, 4, 5 or more) bases are deleted, substituted, inserted and/or added with a promoter activity in the target cell, or (iii) a base sequence not less than 90% (e.g., 90%, 91%, 92%, 93%, 94%, 95%, 96%, 97%, 98%, 99% or above) identical with the base sequence set forth in SEQ ID NO: 187 showing a promoter activity in the target cell.

### (6) Other Base Sequence

Furthermore, the polynucleotide of the present invention may further comprise known sequences such as Polyadenylation (polyA) signal, Kozak consensus sequence and the like besides those mentioned above for the purpose of improving the translation efficiency of mRNA produced by transcription of a base sequence encoding a fusion protein of nuclease-deficient CRISPR effector protein and transcriptional repressor. In addition, the polynucleotide of the present invention may comprise a base sequence encoding a linker sequence, a base sequence encoding NLS and/or a base sequence encoding a tag.

### (7) Exemplified Embodiments of the Present Invention

In one embodiment of the present invention, a polynucleotide is provided comprising:

a base sequence encoding a fusion protein of a nuclease-deficient CRISPR effector protein and a transcriptional repressor,

a promoter sequence for the base sequence encoding the fusion protein of the nuclease-deficient CRISPR effector protein and the transcriptional repressor,

one or two base sequences respectively encoding a guide RNA, wherein the one or two base sequences are selected from a base sequence comprising a sequence set forth in SEQ ID NO: 63, SEQ ID NO: 70, SEQ ID NO: 71, SEQ ID NO: 83, SEQ ID NO: 99, SEQ ID NO: 105, SEQ ID NO: 106, SEQ ID NO: 109, or SEQ ID NO: 111, or the base sequence comprising a sequence set forth in SEQ ID NO: 63, SEQ ID NO: 70, SEQ ID NO: 71, SEQ ID NO: 83, SEQ ID NO: 99, SEQ ID NO: 105, SEQ ID NO: 106, SEQ ID NO: 109, or SEQ ID NO: 111, in which 1 to 3 bases are deleted, substituted, inserted, and/or added, and

a promoter sequence for the base sequence encoding the gRNA,

wherein the nuclease-deficient CRISPR effector protein is dSaCas9 or dSaCas9[-25],

wherein the transcriptional repressor is selected from the group KRAB, MeCP2, SIN3A, HDT1, MBD2B, NIPP1, and HP1A,

wherein the promoter sequence for the base sequence encoding the fusion protein is selected from the group EFS promoter, CMV promoter, CAG promoter, CK8 promoter, myosin heavy chain kinase (MHCK) promoter, muscle creatine kinase (MCK) promoter, synthetic C5-12 (Syn) promoter, and Des promoter, and

wherein the promoter sequence for the base sequence encoding the gRNA is selected from the group U6 promoter, SNR6 promoter, SNR52 promoter, SCR1 promoter, RPR1 promoter, U3 promoter, and H1 promoter.

In one embodiment of the present invention, a polynucleotide is provided comprising:

a base sequence encoding a fusion protein of a nuclease-deficient CRISPR effector protein and a transcriptional repressor,

CK8 promoter for the base sequence encoding the fusion protein of the nuclease-deficient CRISPR effector protein and the transcriptional repressor,

one or two base sequences respectively encoding a guide RNA, wherein the one or two base sequences are selected from a base sequence comprising a sequence set forth in SEQ ID NO: 70, SEQ ID NO: 81, SEQ ID NO: 83, or SEQ ID NO: 99, or a base sequence comprising a sequence set forth in SEQ ID NO: 70, SEQ ID NO: 81, SEQ ID NO: 83, or SEQ ID NO: 99 in which 1 to 3 bases are deleted, substituted, inserted, and/or added, and

U6 promoter for the base sequence encoding the guide RNA,

wherein the nuclease-deficient CRISPR effector protein is dSaCas9, and

wherein the transcriptional repressor is KRAB.

In one embodiment of the present invention, a polynucleotide is provided comprising:

a base sequence encoding a fusion protein of a nuclease-deficient CRISPR effector protein and a transcriptional repressor,

CK8 promoter for the base sequence encoding the fusion protein of the nuclease-deficient CRISPR effector protein and the transcriptional repressor,

a base sequence encoding a guide RNA comprising the base sequence set forth in SEQ ID NO: 83, or the base sequence set forth in SEQ ID NO: 83 in which 1 to 3 bases are deleted, substituted, inserted, and/or added, and

U6 promoter for the base sequence encoding the guide RNA,

wherein the nuclease-deficient CRISPR effector protein is dSaCas9 and

wherein the transcriptional repressor is KRAB.

In an embodiment of the polynucleotide of the present invention, the polynucleotide comprises in order from the 5′ end (i) the base sequence encoding the fusion protein of the nuclease-deficient CRISPR effector protein and the transcriptional repressor and (ii) the base sequence encoding the gRNA. In another embodiment, the polynucleotide comprises in order from the 5′ end (ii) the base sequence encoding the gRNA and (i) the base sequence encoding the fusion protein of the nuclease-deficient CRISPR effector protein and the transcriptional repressor.

### 2. Vector

The present invention provides a vector comprising the polynucleotide of the present invention (hereinafter sometimes referred to as “the vector of the present invention”). The vector of the present invention may be a plasmid vector or a viral vector.

When the vector of the present invention is a plasmid vector, the plasmid vector to be used is not particularly limited and may be any plasmid vector such as cloning plasmid vector and expression plasmid vector. The plasmid vector is prepared by inserting the polynucleotide of the present invention into a plasmid vector by a known method.

When the vector of the present invention is a viral vector, examples of the viral vector to be used include, but are not limited to, adeno-associated virus (AAV) vector, adenovirus vector, lentivirus vector, retrovirus vector, Sendaivirus vector and the like. In the present specification, the “virus vector” or “viral vector” also includes derivatives thereof. Considering the use in gene therapy, AAV vector is preferably used for the reasons such that it can express transgene for a long time, and it is derived from a non-pathogenic virus and has high safety.

A viral vector comprising the polynucleotide of the present invention can be prepared by a known method. In brief, a plasmid vector for virus expression into which the polynucleotide of the present invention has been inserted is prepared, the vector is transfected into an appropriate host cell to allow for transient production of a viral vector comprising the polynucleotide of the present invention, and the viral vector is collected.

In one embodiment of the present invention, when AAV vector is used, the serotype of the AAV vector is not particularly limited as long as the expression of the human DMPK gene in the subject can be repressed, and any of AAV1, AAV2, AAV3, AAV4, AAV5, AAV6, AAV7, AAV8, AAV9, AAVrh. 10 and the like may be used (for the various serotypes of AAV, see, for example, WO 2005/033321 and EP2341068 (A1), which are incorporated herein by reference in their entireties). In another embodiment of the present invention, AAV isolated from monkey (e.g., AAVrh74 (see Mol Ther. 2017 Apr. 5; 25(4): 855-869, etc., which is incorporated herein by reference in its entirety), AAV isolated from porcine (e.g., AAVpol (e.g., see Gene Ther. 2009 November; 16(11): 1320-8, which is incorporated herein by reference in its entirety)), Anc 80, which is a predicted ancestor of AAV1, AAV2, AAV8 and AAV9 (see Cell Rep. 2015 Aug. 11; 12(6):1056-68, which is incorporated herein by reference in its entirety) and the like can also be used as long as the expression of human DMPK gene can be repressed in the subject. Examples of the variants of AAV include, but are not limited to, new serotype with a modified capsid (e.g., WO 2012/057363, which is incorporated herein by reference in its entirety) and the like. For example, in one embodiment of the present invention, a new serotype with a modified capsid improving infectivity for muscle cells can be used, such as AAV₅₈₇MTP, AAV₅₈₈MTP, AAV-B1, AAVM41, and the like (see Yu et al., Gene Ther. 2009 August; 16(8):953-62, Choudhury et al., Mol Ther. 2016 Aug.; 24(7):1247-57, and Yang et al., Proc Natl Acad Sci USA. 2009 Mar. 10; 106(10):3946-51, which are incorporated herein by reference in their entireties).

When an AAV vector is prepared, a known method such as (1) a method using a plasmid, (2) a method using a baculovirus, (3) a method using a herpes simplex virus, (4) a method using an adenovirus, or (5) a method using yeast can be used (e.g., Appl Microbiol Biotechnol. 2018; 102(3): 1045-1054, etc., which is incorporated herein by reference in its entirety). For example, when an AAV vector is prepared by a method using a plasmid, first, a vector plasmid comprising inverted terminal repeat (ITR) at both ends of wild-type AAV genomic sequence and the polynucleotide of the present invention inserted in place of the DNA encoding Rep protein and capsid protein is prepared. On the other hand, the DNA encoding Rep protein and capsid protein which are necessary for forming virus particles are inserted into other plasmids. Furthermore, a plasmid comprising genes (E1A, E1B, E2A, VA and E4orf6) responsible for the helper action of adenovirus necessary for proliferation of AAV is prepared as an adenovirus helper plasmid. The co-transfection of these three kinds of plasmids into the host cell causes the production of recombinant AAV (i.e., AAV vector) in the cell. As the host cell, a cell capable of supplying a part of the gene products (proteins) of the genes responsible for the aforementioned helper action (e.g., 293 cell etc.) is preferably used. When such cell is used, it is not necessary to carry the gene encoding a protein that can be supplied from the host cell in the aforementioned adenoviral helper plasmid. The produced AAV vector is present in the culture medium and/or cell. Thus, a desired AAV vector is prepared by collection of the virus from the culture medium after destroying the host cell with freeze-thawing or the like and then subjecting the virus fraction to separation and purification by density gradient ultracentrifugation method using cesium chloride, column method or the like.

AAV vector has great advantages in terms of safety, gene transduction efficiency and the like, and is used for gene therapy. However, it is known that the size of polynucleotide that can be packaged is limited. For example, in one embodiment of the present invention, the full-length including the base length of the polynucleotide comprising a base sequence encoding a fusion protein of dSaCas9 and KRAB, a base sequence encoding gRNA targeting an expression regulatory region of human DMPK gene, and CK8 promoter sequence and U6 promoter sequence as the promoter sequences, and the ITR region is about 4.9 kb and the polynucleotide can be carried in a single AAV vector.

### 3. Pharmaceutical Composition for Treating or Preventing DM1

The present invention also provides a pharmaceutical composition comprising the polynucleotide of the present invention or the vector of the present invention (hereinafter sometimes referred to as “the pharmaceutical composition of the present invention”). The pharmaceutical composition of the present invention can be used for treating or preventing DM1.

The pharmaceutical composition of the present invention comprises the polynucleotide of the present invention or the vector of the present invention as an active ingredient, and may be prepared as a formulation comprising such active ingredient (i.e., the polynucleotide of the present invention or the vector of the present invention) and, generally, a pharmaceutically acceptable carrier.

In an embodiment, the pharmaceutical composition of the present invention is administered parenterally, and may be administered topically or systemically. The pharmaceutical composition of the present invention can be administered by, but are not limited to, for example, intravenous administration, intraarterial administration, subcutaneous administration, intraperitoneal administration, or intramuscular administration.

The dose of the pharmaceutical composition of the present invention to a subject is not particularly limited as long as it is an effective amount for the treatment and/or prevention. It may be appropriately optimized according to the active ingredient, dosage form, age and body weight of the subject, administration schedule, administration method and the like.

In one embodiment of the present invention, the pharmaceutical composition of the present invention can be not only administered to the subject affected with DM1 but also prophylactically administered to subjects who may develop DM1 in the future based on the genetic background analysis and the like. The term “treatment” in the present specification also includes remission of disease, in addition to the cure of diseases. In addition, the term “prevention” may also include delaying the onset of disease, in addition to prophylaxis of the onset of disease. The pharmaceutical composition of the present invention can also be referred to as “the agent of the present invention” or the like.

### 4. Method for Treatment or Prevention of DM1

The present invention also provides a method for treating or preventing DM1, comprising administering the polynucleotide of the present invention or the vector of the present invention to a subject in need thereof (hereinafter sometimes referred to as “the method of the present invention”). In addition, the present invention includes the polynucleotide of the present invention or the vector of the present invention for use in the treatment or prevention of DM1. Furthermore, the present invention includes use of the polynucleotide of the present invention or the vector of the present invention in the manufacture of a pharmaceutical composition for the treatment or prevention of DM1.

The method of the present invention can be practiced by administering the aforementioned pharmaceutical composition of the present invention to a subject affected with DM1, and the dose, administration route, subject and the like are the same as those mentioned above.

Measurement of the symptoms may be performed before the start of the treatment using the method of the present invention and at any timing after the treatment to determine the response of the subject to the treatment.

The method of the present invention can improve, but are not limited to, any symptom of DM1 such as the function of skeletal muscle and/or cardiac muscle. Muscles or tissue to be improved in the function thereof are not particularly limited, and any muscles and tissue, and muscle groups can be mentioned.

### 5. Ribonucleoprotein

The present invention provides a ribonucleoprotein comprising the following (hereinafter sometimes referred to as “RNP of the present invention”):

(c) a fusion protein of a nuclease-deficient CRISPR effector protein and a transcriptional repressor, and

(d) a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene.

As the nuclease-deficient CRISPR effector protein, transcriptional repressor, and guide RNA comprised in the RNP of the present invention, the nuclease-deficient CRISPR effector protein, transcriptional repressor, and guide RNA explained in detail in the above-mentioned section of “1. Polynucleotide” can be used. The fusion protein of nuclease-deficient CRISPR effector protein and transcriptional repressor to be comprised in the RNP of the present invention can be produced by, for example, introducing a polynucleotide encoding the fusion protein into the cell, bacterium, or other organism to allow for the expression, or an in vitro translation system by using the polynucleotide. In addition, guide RNA comprised in the RNP of the present invention can be m produced by, for example, chemical synthesis or an in vitro transcription system by using a polynucleotide encoding the guide RNA. The thus-prepared fusion protein and guide RNA are mixed to prepare the RNP of the present invention. Where necessary, other substances such as gold particles may be mixed. To directly deliver the RNP of the present invention to the target cell, tissue and the like, the RNP may be encapsulated in a lipid nanoparticle (LNP) or loaded in an extracellular vesicle by a known method. The RNP of the present invention can be introduced into the target cell, tissue and the like by a known method. For example, Lee K., et al., Nat Biomed Eng. 2017; 1:889-901, WO 2016/153012 and the like can be referred to for encapsulation in LNP and introduction method, which are incorporated herein by reference in their entireties.

In one embodiment of the present invention, the guide RNA comprised in RNP of the present invention targets continuous 18 to 24 nucleotides in length, preferably 18 to 23 nucleotides in length, more preferably 18 to 22 nucleotides in length, in the following region: 45,778,884-45,783,985 existing in the GRCh38.p12 position of human chromosome 19 (Chr 19).

In one embodiment, the guide RNA targets a base sequence of continuous 18 to 24 nucleotides in length, preferably 18 to 23 nucleotides in length, more preferably 18 to 22 nucleotides in length, in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119. In another embodiment, the guide RNA targets a base sequence of continuous 18 to 24 nucleotides in length, preferably 18 to 23 nucleotides in length, more preferably 18 to 22 nucleotides in length, in a region set forth in SEQ ID NO: 127, SEQ ID NO: 46, SEQ ID NO: 128, SEQ ID NO: 66, SEQ ID NO: 68, SEQ ID NO: 130, SEQ ID NO: 131, SEQ ID NO: 132, SEQ ID NO: 88, SEQ ID NO: 91, SEQ ID NO: 96, SEQ ID NO: 99, SEQ ID NO: 100, SEQ ID NO: 134, SEQ ID NO: 108, SEQ ID NO: 109, SEQ ID NO: 111, SEQ ID NO: 117, or SEQ ID NO: 119. In a still another embodiment, the guide RNA targets a base sequence of continuous 18 to 24 nucleotides in length, preferably 18 to 23 nucleotides in length, more preferably 18 to 22 nucleotides in length, in a region set forth in SEQ ID NO: 63, SEQ ID NO: 136, SEQ ID NO: 83, SEQ ID NO: 99, SEQ ID NO: 135, SEQ ID NO: 109, or SEQ ID NO: 111. In a yet another embodiment, the guide RNA targets a region comprising the whole or a part of the sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117, or SEQ ID NO: 119. In another embodiment of the present invention, the guide RNA targets a region comprising the whole or a part of the sequence set forth in SEQ ID NO: 63, SEQ ID NO: 70, SEQ ID NO: 71, SEQ ID NO: 83, SEQ ID NO: 99, SEQ ID NO: 105, SEQ ID NO: 106, SEQ ID NO: 109, or SEQ ID NO: 111. In still another embodiment of the present invention, the guide RNA targets a region comprising the whole or a part of the sequence set forth in SEQ ID NO: 70, SEQ ID NO: 81, SEQ ID NO: 83, or SEQ ID NO: 99. In one embodiment of the present invention, the guide RNA targets a region comprising the whole or a part of the sequence set forth in SEQ ID NO: 83.

In one embodiment of the present invention, the guide RNA comprising the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID. NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186, or the base sequence set forth in SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, or SEQ ID NO: 186 in which 1 to 3 bases are deleted, substituted, inserted, and/or added respectively can be used. In one embodiment of the present invention, the guide RNA comprising the base sequence set forth in SEQ ID NO: 161, SEQ ID NO: 164, SEQ ID NO: 165, SEQ ID NO: 171, SEQ ID NO: 177, SEQ ID NO: 180, SEQ ID NO: 181, SEQ ID NO: 183, or SEQ ID NO: 184, or the base sequence set forth in SEQ ID NO: 161, SEQ ID NO: 164, SEQ ID NO: 165, SEQ ID NO: 171, SEQ ID NO: 177, SEQ ID NO: 180, SEQ ID NO: 181, SEQ ID NO: 183, or SEQ ID NO: 184 in which 1 to 3 bases are deleted, substituted, inserted, and/or added respectively can be used. In another embodiment of the present invention, the guide RNA comprising the base sequence set forth in SEQ ID NO: 164, SEQ ID NO: 169, SEQ ID NO: 171, or SEQ ID NO: 177, or the base sequence set forth in SEQ ID NO: 164, SEQ ID NO: 169, SEQ ID NO: 171, or SEQ ID NO: 177 in which 1 to 3 bases are deleted, substituted, inserted, and/or added respectively can be used. In still another embodiment of the present invention, the guide RNA comprising the base sequence set forth in SEQ ID NO: 171, or the base sequence set forth in SEQ ID NO: 171 in which 1 to 3 bases are deleted, substituted, inserted, and/or added respectively can be used.

### 6. Others

The present invention also provides a composition or kit comprising the following for repression of the expression of the human DMPK gene:

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene, or a polynucleotide encoding the guide RNA.

The present invention also provides a method for treating or preventing myotonic dystrophy type 1, comprising a step of administering the following (e) and (f):

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene, or a polynucleotide encoding the guide RNA.

The present invention also provides use of the following (e) and (f):

(e) a fusion protein of a nuclease-deficient CRISPR effector protein and a transcriptional repressor, or a polynucleotide encoding the fusion protein, and

(f) a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene, or a polynucleotide encoding the guide RNA,

in the manufacture of a pharmaceutical composition for the treatment or prevention of DM1.

As the nuclease-deficient CRISPR effector protein, transcriptional repressor, guide RNA, as well as polynucleotides encoding them and vectors in which they are carried in these inventions, those explained in detail in the above-mentioned sections of “1. Polynucleotide”, “2. Vector” and “5. Ribonucleoprotein” can be used. The dose, administration route, subject, formulation and the like of the above-mentioned (e) and (f) are the same as those explained in the section of “3. Pharmaceutical composition for treating or preventing DM1”.

Other features of the invention will become apparent in the course of the following descriptions of exemplary embodiments which are given for illustration of the invention and are not intended to be limiting thereof.

### EXAMPLES

### Example 1. Screening of gRNAs for Human DMPK Gene Using, iCM and iDM Cells

### (1) Experimental Methods

### Selection of DMPK Targeting Sequences

Roughly 7.4 kb of sequence around the promoter region of the human DMPK gene (Chr19: GRCh38.p12; 45,777,342-45,784,715) was scanned for sequences that can be targeted by a nuclease-deficient SaCas9 (D10A and N580A mutant; dSaCas9 (SEQ ID NO: 139)) complexed with gRNA, defined herein as a targeting sequence. Targeting sequences were initially specified by the 19-21-nucleotide segment adjacent to a protospacer adjacent motif (PAM) having the sequence NNGRRT (5′-19-21 nt targeting sequence-NNGRRT-3′), and were filtered to include only those with a perfect match (targeting sequence and PAM sequences) for the corresponding region of the cynomolgus monkey (Macaca fascicularis) genome (listed as “TRUE” in Table 1). Additional 21-nucleotide targeting sequences were also selected that direct RNP to regions that exhibit high DNase sensitivity in DNase-Seq experiments curated by The ENCODE Project (The ENCODE Project Consortium, Nature. 2012 Sep. 6; 489(7414): 57-74; https://www.encodeproject.org).

Construction of Lentiviral Transfer Plasmid (pED162)

pLentiCRISPR v2 was purchased from Genscript (https://www.genscript.com) and the following modifications were made: the SpCas9 gRNA scaffold sequence was replaced by SaCas9 gRNA scaffold sequence (SEQ ID NO: 150); SpCas9 was replaced with dSaCas9 fused to Kruppel-associated box transcriptional repression domains (KRAB) with two NLSes sandwiching dSaCas9 (SV40 NLS-dSaCas9-NLS-KRAB [SEQ ID NO: 151 (DNA) and 152 (Protein)]); and the puroR cassette was replaced by a blastR cassette [SEQ ID NO: 153 (DNA) and SEQ ID NO: 154 (Protein)]. dSaCas9 was attached with two nuclear localization signal (NLS) in its N-terminus (amino acid sequence shown by SEQ ID NO: 188, DNA sequence shown by SEQ ID NO: 189) and C-terminus (amino acid sequence shown by SEQ ID NO: 190, DNA sequence shown by SEQ ID NO: 191) to enable efficient localization of the effector molecules to the nucleus. KRAB can repress gene expression when localized to promoters by inhibiting transcription (Gilbert L A, et al., Cell, 2013 Jul. 18; 154(2):442-51). KRAB was tethered to the C-terminus of dSaCas9 (D10A and N580A mutant), which is referred to as dSaCas9-KRAB hereinafter, and targeted to human DMPK promoter regions as directed by targeting sequences (FIG. 1). The generated plasmid was named pED162.

gRNA Cloning

Three control non-targeting targeting sequences (Table 1, SEQ ID NOs: 1 through 3) and 123 targeting sequences (Table 1, SEQ ID NOs: 4 through 126) were cloned into pED162. Forward and reverse oligos were synthesized by Integrated DNA Technologies in the following format: Forward; 5′ CACC(G)-19-21 basepair targeting sequence-3′, and Reverse: 5′ AAAC—19-21 basepair reverse complement targeting sequence-(C)-3′, where bases in parenthesis were added if the target did not begin with a G. Oligos were resuspended in Tris-EDTA buffer (pH 8.0) at 100 μM. 1.5 μl of each complementary oligo were combined in a 50 μl reaction in NE Buffer 3.1 (New England Biolabs (NEB) #B7203S). The reaction was heated to 95° C. in 1 L H₂O and allowed to cool to 25° C., thus annealing oligos with sticky end overhangs compatible with cloning to pED162. Annealed oligos were combined with lentiviral transfer plasmid pED162 which had been digested with BsmBI and gel purified, and ligated with T4 DNA ligase (NEB #M0202S) according to manufacturer's protocol. 2 μl of the ligation reaction was transformed to NEB® Stable Competent cells (NEB #C30401) according to the manufacturer's protocol. The resulting construct drives expression of sgRNAs comprising crRNA encoded by individual targeting sequences fused to their 3′ end with tracrRNA (GUUUUAGUACUCUGGAAACAGAAUCUACUAAAACAAGGCAAAAUGCCGUGUUUAUCACGUC AACUUGUUGGCGAGAUUUUUU) (SEQ ID NO: 156), which is encoded from the SaCas9 gRNA scaffold sequence added with a termination signal of U6 polymerase TTTTTT, by a U6 promoter (SEQ ID NO: 155).

### Lentivirus Generation

Lenti-Pac 293Ta Cell Line (Genecopoeia #LT008) was seeded at 0.8-1.0×10⁶ cells/well in 6 well cell culture dishes (VWR #10062-892) in 2 ml growth medium (DMEM media supplemented with 10% FBS and 2 mM fresh L-glutamine, 1 mM sodium pyruvate and MEM Non-Essential Amino Acids (Thermo Fisher #11140050)) and incubated at 37° C./5% CO₂ for 24 hours. The next day TransIT-VirusGEN® transfection reactions (Mirus Bio #MIR6700) were set up according to manufacturer's protocol With 1.5 μg packaging plasmid mix [lug packaging plasmid (pCMV delta R8.2; addgene Plasmid #12263) and 0.5 μg envelope expression plasmid (pCMV-VSV-G; addgene Plasmid #8454)] and 1 μg of transfer plasmid pED162 containing sequence encoding dSaCas9-KRAB and indicated sgRNAs. Lentivirus was harvested 48 hours following transfection by passing media supernatant through a 0.45 μm PES filter (VWR #10218-488).

### Transduction of iCM and iDM Cells

Immortalized non-DM control (Ctrl) myoblast (termed iCM) and immortalized DM1 myoblast (termed iDM) were obtained from Institut de Myologie which established these cell lines by the methods described in Dis Model Mech. 2017 Apr. 1; 10(4):487-497, which is incorporated herein by reference in its entirety. For transduction, cells were seeded at 0.05×10⁶ cells/well in 12 well cell culture dishes (VWR #10062-894) in 1 ml medium containing growth medium [PromoCell Skeletal Muscle Cell Growth Medium Kit; part number: C-23160 (note: media was supplemented with 20% FBS, rather than 5% as directed by kit, and 30 μg/ml Gentamicin S)] and incubated at 37° C./5% CO₂ for 24 hours. The next day, the medium was replaced with 1 ml growth medium supplemented with 10 μg/ml Polybrene (Sigma #TR-1003-G) and 0.3 ml lentivirus supernatant (see above) corresponding to each sgRNA comprising crRNA encoded by individual targeting sequences (Table 1) fused with tracrRNA was added to each well. Cells were incubated with lentivirus for 48 hours before viral media was removed and replaced with selection media [growth media supplemented with 10 μg/ml Blasticidin (Thermo Fisher #A1113903)]. Following 48 hours of incubation in selection media one third of cells were passed into new wells (from 12 well plates) in growth media. After allowing cells to seed for 24 hours, growth media were replaced with selection media. Following 48 hours of culture in selection media, cells were harvested and RNA was extracted with RNeasy® 96 kit (Qiagen #74182) as directed by the manufacturer.

### Gene Expression Analysis

For gene expression analysis, CDNA was generated from 0.2 μg of total RNA according to High-Capacity cDNA Reverse Transcription Kit (Thermo Fisher #4368813) protocol in a 20 μl volume. cDNA was diluted 10-fold and analyzed using Taqman™ Fast Advanced Master Mix (Thermo Fisher #4444557) according to the manufacturer's protocol. Taqman probes (DMPK: Assay Id Hs01094336 ml FAM; HPRT: Assay Id Hs99999909 ml VIC_PL) were obtained from Thermo Fisher. Taqman probe-based real-time PCR reactions were processed and analyzed by QuantStudio 5 Real-Time PCR system as directed by Taqman Fast Advanced Master Mix protocol.

### Data Analysis

For each sample and three controls, deltaCt values were calculated by subtracting the average Ct values from 3 technical replicates of the HPRT probe from the DMPK probe (Average Ct DMPK—Average Ct HPRT). Expression values were determined for each sample using the formula 2^{-(deltaCt)} Sample expression values (Table 1; SEQ ID NOs: 4-126) were then normalized to the average of 3 control expression values (Table 1; SEQ ID NOs: 1 to 3) for each experiment to determine the relative DMPK expression for each sample. Two biological replicates for each cell line were analyzed and the average from all the experiments was calculated (Table 1).

### (2) Results

### Repression of DMPK Gene Expression by the RNP

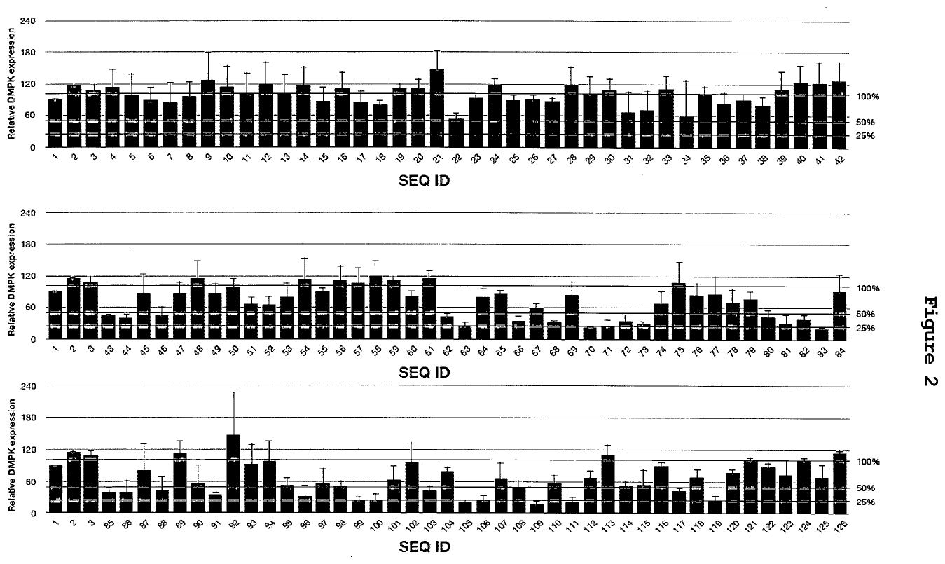

Lentivirus was produced that deliver expression cassettes for dSaCas9-KRAB and sgRNAs for each targeting sequence to iCM and iDM cells. Transduced cells were selected for resistance to blasticidin, and DMPK expression was quantitated using the Taqman Assay (Table 1). Expression values from each sample were normalized to an average of DMPK expression in cells transduced with control sgRNAs (Table 1; SEQ ID NOs: 1, 2 and 3). Average expression levels were measured across duplicates of iCM and iDM cell lines (Table 1; Average DMPK ALL and FIG. 2).

Table 1 Targeting sequences used to screen expression regulatory region of DMPK gene

TABLE 1

| | | | plus | | | | | | | |
|---|---|---|---|---|---|---|---|---|---|---|
| | Coordinate | | or | | | iCM_RE_ | iCM_RE_ | iDM_RE_ | iDM_RE_ | Average |
| SEQ | (Chr19: | nt | minus | | Cyno- | Rep1_ | Rep2_ | Rep1_ | Rep2_ | DMPK |
| ID | GRCh38.p12) | length | strand | Sequence (5′→3′) | Match | DMPK | DMPK | DMPK | DMPK | ALL |
| 1 | NA | 20 | — | acggaggctaagcgtcgcaa | TRUE | 87.02 | 87.02 | 91.11 | 91.11 | 89.06 |
| 2 | HA | 20 | — | cgcttccgcggcccgttcaa | TRUE | 113.39 | 113.39 | 115.89 | 115.89 | 114.64 |
| 3 | NA | 20 | — | gtaggcgcgccgctctctac | TRUE | 97.39 | 97.39 | 116.90 | 116.90 | 107.14 |
| 4 | 45777464 | 21 | −1 | cccttcttctttggcctcgac | TRUE | 138.8 | 73.27 | 97.97 | 144.53 | 113.64 |
| 5 | 45777440 | 21 | 1 | cccagtcgaggccaaagaaga | TRUE | 120.25 | 39.62 | 103.63 | 129.09 | 98.15 |
| 6 | 45777515 | 21 | −1 | cccccggagacacggctgggc | TRUE | 99.27 | 55.29 | 113.56 | 81.75 | 87.47 |
| 7 | 45777507 | 21 | 1 | ctccgggggacacagcaaccg | TRUE | 96.04 | 39.70 | 67.89 | 128.91 | 83.13 |
| 8 | 45777800 | 21 | −1 | ctgggacaggcagctacgggc | TRUE | 117.97 | 62.26 | 80.33 | 119.89 | 95.11 |
| 9 | 45777799 | 21 | 1 | aggcccaccgcccacagcctg | TRUE | 195.53 | 68.11 | 105.18 | 131.07 | 124.97 |
| 10 | 45777823 | 21 | 1 | gatctcgggggacaggtagtc | TRUE | 149.35 | 68.70 | 92.68 | 144.93 | 113.91 |
| 11 | 45777934 | 21 | −1 | gcccagagctggtgggcccag | TRUE | 128.08 | 62.02 | 73.11 | 139.73 | 100.74 |
| 12 | 45776175 | 21 | 1 | gatgtggccacagcggtccag | TRUE | 127.90 | 70.42 | — | 154.76 | 117.69 |
| 13 | 45778185 | 21 | 1 | cagcggtccagcaggatgttg | TRUE | 110.91 | 66.59 | 80.54 | 146.00 | 101.01 |
| 14 | 45778470 | 21 | −1 | gatagcaagcttgttccctgg | TRUE | 148.46 | 77.17 | 87.79 | 145.43 | 114.71 |
| 15 | 45778549 | 21 | 1 | gtagaagcgcgccatctcggc | TRUE | 71.09 | 69.25 | 78.23 | 125.77 | 86.08 |
| 16 | 45778600 | 21 | −1 | actgctgagcaagtttgggga | TRUE | 62.07 | 116.61 | 130.87 | 131.02 | 110.14 |
| 17 | 45778652 | 21 | −1 | ccccttctcagtacctggtca | TRUE | 113.82 | 67.08 | 86.42 | 61.15 | 82.12 |
| 18 | 45778627 | 21 | 1 | ctccatgaccaggtactgaga | TRUE | 92.43 | 73.49 | 79.85 | 70.43 | 79.05 |
| 19 | 45776638 | 21 | 1 | ggtactgagaaggggttcgtc | TRUE | 122.63 | 98.65 | 108.90 | 111.73 | 110.48 |
| 20 | 45778758 | 21 | −1 | tggggctgcaggtctctgccc | TRUE | 89.95 | 128.02 | 122.31 | 100.95 | 110.31 |
| 21 | 45778902 | 21 | −1 | tcccagaacatctcaggggga | TRUE | 183.05 | 102.82 | 165.03 | 136.15 | 146.76 |
| 22 | 45778906 | 21 | −1 | tctttcccagaacatctcagg | TRUE | 58.11 | 64.67 | 49.83 | 39.37 | 52.99 |
| 23 | 45778946 | 21 | −1 | ctgggacctattctctgggtg | TRUE | 92.68 | 83.80 | 99.29 | 87.70 | 90.87 |
| 24 | 45778953 | 21 | −1 | ggagtgtctgggacctattct | TRUE | 109.17 | 124.48 | 129.33 | 95.68 | 114.671 |
| 25 | 45778975 | 21 | −1 | aacggggccactgcctaggga | TRUE | 94.69 | 95.11 | 89.77 | 73.35 | 88.23 |
| 26 | 45778955 | 20 | 1 | tccctaggcagtggccccgt | TRUE | 88.30 | 92.69 | 79.72 | 98.60 | 89.83 |
| 27 | 45779018 | 20 | −1 | ggggggtctcttacctggaa | TRUE | 88.10 | 95.30 | 82.72 | 79.91 | 86.51 |
| 28 | 45779108 | 21 | −1 | ggggccttccctatctgagac | FALSE | 143.89 | 69.50 | 142.21 | 111.02 | 116.65 |
| 29 | 45779137 | 21 | −1 | tgcggtaagagggtgtgtcag | FALSE | 120.88 | 53.88 | 126.17 | 98.73 | 99.91 |
| 30 | 45779150 | 21 | −1 | gggaaagtctgtgtgcggtaa | FALSE | 107.77 | 73.33 | 124.12 | 121.33 | 106.64 |
| 31 | 45779196 | 21 | −1 | gggagaggaagctctaggatt | TRUE | 117.72 | 27.13 | 65.48 | 55.27 | 66.40 |
| 32 | 45779203 | 21 | −1 | ggaggtggggagaggaagctc | TRUE | 104.85 | 21.91 | 54.68 | 95.18 | 69.16 |
| 33 | 45779271 | 21 | −1 | actacctggtgagctccgggc | TRUE | 122.88 | 85.40 | 138.00 | 94.83 | 110.28 |
| 34 | 45779330 | 21 | −1 | gttggtgaatggggaccggcg | TRUE | — | 12.04 | 24.88 | 137.55 | 58.16 |
| 35 | 45779347 | 21 | −1 | cgtgaggagagggacgtgttg | TRUE | 119.05 | 91.71 | 106.22 | 91.81 | 102.20 |
| 36 | 45779522 | 21 | 1 | cctgaggtcgagatagtgaga | FALSE | 79.64 | 54.05 | 94.91 | 100.06 | 82.17 |
| 37 | 45779608 | 21 | −1 | tgggtgcagaagcgggtggag | FALSE | 86.59 | 72.53 | 99.66 | 98.20 | 89.24 |
| 38 | 45779613 | 21 | −1 | acggctgggtgcagaagcggg | FALSE | 87.68 | 78.74 | 58.35 | 94.72 | 79.87 |
| 39 | 45779720 | 21 | −1 | ggggcggggccttagaaattg | FALSE | 157.32 | 77.19 | 101.23 | 107.10 | 110.71 |
| 40 | 45779748 | 21 | −1 | gactcgtggtgggcggggcat | FALSE | 150.18 | 78.33 | 144.03 | 121.12 | 123.41 |
| 41 | 45779721 | 21 | 1 | aacccctatgccccgcccacc | FALSE | 132.60 | 74.37 | 168.61 | 110.00 | 121.39 |
| 42 | 45779787 | 21 | 1 | cgccccgcgtccgatcacctt | TRUE | 141.13 | 78.50 | 162.94 | 116.70 | 124.82 |
| 43 | 45779878 | 19 | 1 | gtggtgggggaaaagaacc | TRUE | 45.08 | 48.99 | 45.68 | 45.89 | 46.41 |
| 44 | 45779966 | 21 | −1 | agagagaaggggagacagaca | FALSE | 42.68 | 24.74 | 47.03 | 39.60 | 38.51 |
| 45 | 45780114 | 21 | 1 | tccaagggtgtgcaggatggt | FALSE | 138.00 | 49.69 | 77.81 | 82.81 | 87.08 |
| 46 | 45780119 | 21 | 1 | gggtgtgcaggatggttaggg | FALSE | 42.45 | 24.16 | 42.40 | 66.67 | 43.92 |
| 47 | 45780127 | 21 | 1 | aggatggttagggtggggtaa | FALSE | 110.52 | 59.01 | 88.97 | 88.04 | 86.64 |
| 48 | 45780194 | 21 | −1 | tttccttctccccttgttctt | TRUE | 125.85 | 65.70 | 144.19 | 124.99 | 115.18 |
| 49 | 45780225 | 21 | −1 | tcagggtggaaggtggaagaa | FALSE | 87.95 | 58.19 | 95.31 | 101.33 | 85.69 |
| 50 | 45780244 | 20 | −1 | cctgaactgtcacctggagt | TRUE | 104.19 | 90.84 | 79.54 | 117.75 | 98.08 |
| 51 | 45780244 | 20 | −1 | cctgaactgtcacctggagt | TRUE | 70.57 | 82.39 | 55.60 | 55.60 | 66.04 |
| 52 | 45780251 | 21 | −1 | ggctgcacctgaactgtcacc | FALSE | 63.71 | 49.98 | 55.08 | 88.96 | 64.43 |
| 53 | 45780286 | 21 | −1 | ggacattctacatgagaacgt | FALSE | 71.53 | 64.93 | 62.82 | 119.58 | 79.72 |
| 54 | 45780256 | 21 | 1 | ccccacccccacgttctcatg | FALSE | 110.55 | 59.09 | 120.30 | 160.26 | 112.55 |
| 55 | 45780267 | 19 | 1 | cgttctcatgtagaatgtc | TRUE | 94.64 | 96.41 | 89.90 | 75.60 | 89.14 |
| 56 | 45780284 | 21 | 1 | tcctgggtaacggcccagacg | FALSE | 120.72 | 78.62 | 145.16 | 97.50 | 110.50 |
| 57 | 45780355 | 21 | −1 | tggttcaagaaccacctgcat | FALSE | 131.59 | 62.02 | 108.62 | 119.04 | 105.32 |
| 58 | 45780349 | 21 | 1 | tgaaccacactttggaaaacc | FALSE | 139.28 | 108.34 | 145.53 | 80.53 | 118.42 |
| 59 | 45780519 | 21 | −1 | cctgattgtcatctgtaaata | TRUE | 109.23 | 110.74 | 120.19 | 97.35 | 109.38 |
| 60 | 45780526 | 21 | 1 | agaagtcaaggtcctatgact | TRUE | 69.46 | 88.66 | 90.23 | 77.02 | 81.34 |
| 61 | 45780618 | 21 | 1 | gacagggaggcctggactccc | TRUE | 123.54 | 114.65 | 127.61 | 94.15 | 114.98 |
| 62 | 45780650 | 21 | 1 | aagtggaggcaggatggagag | TRUE | 50.95 | 39.97 | 40.95 | 40.91 | 43.20 |
| 63 | 45780654 | 21 | 1 | ggaggcaggatggagagaggg | TRUE | 12.64 | 31.01 | 20.01 | 29.32 | 23.25 |
| 64 | 45780867 | 21 | −1 | gcctggggcgggggagggggc | TRUE | 62.65 | 103.41 | 70.14 | 79.23 | 78.86 |
| 65 | 45780888 | 21 | 1 | agatgcagctcgggccacaaa | TRUE | 81.20 | 94.72 | 86.48 | 80.35 | 85.68 |
| 66 | 45780967 | 21 | −1 | gcagggtaaactgagaccggg | TRUE | 29.66 | 22.91 | 49.61 | 30.58 | 33.19 |
| 67 | 45781030 | 21 | 1 | gggagaaggtttttccagagg | TRUE | 64.97 | 65.44 | 48.21 | 58.86 | 59.37 |
| 68 | 45781243 | 21 | 1 | gacatatgagggccagagggg | FALSE | 32.32 | 24.32 | 33.78 | 35.39 | 31.46 |
| 69 | 45781252 | 21 | 1 | gggccagaggggcagggtgct | TRUE | 84.09 | 66.39 | 119.69 | 62.95 | 83.28 |
| 70 | 45781282 | 21 | 1 | agaaaccagtgaccagtgagc | TRUE | 23.14 | 20.64 | 23.21 | 21.86 | 22.22 |
| 71 | 45781387 | 21 | −1 | gcctggggccagggctggaag | TRUE | 20.97 | 10.79 | 40.92 | 25.22 | 24.48 |
| 72 | 45781498 | 21 | −1 | tcagccaccattcccgccacc | FALSE | 23.89 | 44.36 | 21.25 | 45.84 | 33.84 |
| 73 | 45781524 | 21 | −1 | gtctcggttcctatgagccgt | FALSE | 27.57 | 26.85 | 24.56 | 36.90 | 28.97 |
| 74 | 45781519 | 21 | 1 | cgagactttggggctgggggt | TRUE | 73.14 | 34.44 | 75.45 | 88.95 | 67.99 |
| 75 | 45781523 | 21 | 1 | actttggggctgggggtgggg | TRUE | 114.18 | 57.79 | 101.92 | 155.65 | 107.39 |
| 76 | 45781602 | 21 | −1 | gagggacaactttgggttctt | TRUE | 85.52 | 58.90 | 74.19 | 114.90 | 83.38 |
| 77 | 45781582 | 21 | 1 | aagaacccaaagttgtccctc | TRUE | 120.44 | 42.36 | 70.41 | 105.09 | 84.58 |
| 78 | 45781648 | 21 | 1 | tggttctcccacagggcccgc | FALSE | 104.83 | 47.04 | 54.25 | 71.38 | 69.38 |
| 79 | 45781819 | 21 | 1 | catagagcccacttttggggg | FALSE | 89.86 | 74.17 | 85.96 | 56.05 | 76.51 |
| 80 | 45781967 | 21 | −1 | tgtgactcagagccatggctt | FALSE | 32.90 | 31.73 | 46.30 | 59.56 | 42.62 |
| 81 | 45781937 | 21 | 1 | tgtgactcctaagccatggct | TRUE | 52.83 | 14.62 | 30.71 | 22.30 | 30.11 |
| 82 | 45781965 | 19 | −1 | tgactcagagccatggctt | TRUE | 43.14 | 45.50 | 25.17 | 38.77 | 38.14 |
| 83 | 45781997 | 21 | −1 | cagagtaaggtcagcagaggc | TRUE | 18.87 | 15.39 | 23.48 | 22.27 | 20.00 |
| 84 | 45781983 | 21 | 1 | gctgaccttactctgcccctc | TRUE | 127.49 | 57.38 | 69.11 | 108.98 | 90.74 |
| 85 | 45782175 | 19 | −1 | gggctcctgcagatggggt | TRUE | 44.15 | 43.85 | 44.43 | 24.14 | 39.14 |
| 86 | 45782213 | 21 | −1 | tggccgacttcttgcagtggg | TRUE | 71.85 | 22.88 | 38.12 | 21.26 | 38.53 |
| 87 | 45782217 | 21 | −1 | tacgtggccgacttcttgcag | TRUE | 92.60 | 114.52 | 110.26 | 9.39 | 81.69 |
| 88 | 45782280 | 21 | 1 | ggggctccagccccaggaagc | TRUE | 76.77 | 14.27 | 36.77 | 42.05 | 42.47 |
| 89 | 45782374 | 21 | 1 | cccctccccgggccgggggct | TRUE | 130.26 | 77.49 | 110.57 | 131.13 | 112.36 |
| 90 | 45782563 | 21 | −1 | ggcgggaggggggctggacca | TRUE | 100.09 | 18.98 | 52.22 | 56.49 | 56.95 |
| 91 | 45782651 | 21 | −1 | attccccctactctagcactg | FALSE | 29.90 | 29.88 | 39.24 | 38.95 | 34.49 |
| 92 | 45782707 | 21 | −1 | ctctgtgctctacctttttat | TRUE | 121.48 | 65.22 | 146.53 | 256.57 | 147.45 |
| 93 | 45782805 | 21 | −1 | ggagcaagggggtggttgcta | TRUE | — | 54.40 | 97.95 | 126.58 | 92.98 |
| 94 | 45782702 | 21 | 1 | acagaggaagccacaggtgtg | TRUE | 138.42 | 60.02 | 96.44 | — | 98.29 |
| 95 | 45782893 | 19 | 1 | gggaggccagggcagcttc | TRUE | 72.39 | 48.88 | 33.91 | 52.60 | 51.94 |
| 96 | 45782906 | 21 | 1 | agcttcttgggtgactcagag | TRUE | 54.89 | 8.50 | 30.23 | — | 31.21 |
| 97 | 45782945 | 21 | −1 | tgtccgagtcggaatccatct | TRUE | 70.35 | 25.78 | 48.04 | 86.59 | 57.69 |
| 98 | 45782957 | 21 | −1 | gctaaatttaactgtccgagt | TRUE | 59.16 | 43.62 | 40.66 | 60.66 | 51.03 |
| 99 | 45782963 | 21 | −1 | ctgagggctaaatttaactgt | TRUE | 20.26 | 17.60 | 22.85 | 34.48 | 23.80 |
| 100 | 45783081 | 21 | −1 | ggtcacccctgttcaggctct | TRUE | 22.62 | 9.80 | 29.57 | 38.39 | 25.09 |
| 101 | 45783051 | 21 | 1 | gggcaccctcagagcctgaac | TRUE | 97.13 | 33.39 | 52.77 | 66.97 | 62.57 |
| 102 | 45783167 | 21 | −1 | ccaggggccaaggagctattt | TRUE | 119.69 | 54.63 | 75.27 | 134.51 | 96.02 |
| 103 | 45783206 | 21 | −1 | gtttctaatcccagcctgggc | TRUE | 49.83 | 27.77 | 43.30 | 49.19 | 42.52 |
| 104 | 45783174 | 20 | 1 | agccacattcctgcccaggc | TRUE | 80.51 | 64.79 | 81.96 | 86.44 | 78.42 |
| 105 | 45783198 | 21 | 1 | attagaaacagaaacatttcg | TRUE | 24.86 | 14.08 | 21.43 | 22.78 | 20.79 |
| 106 | 45783205 | 21 | 1 | acagaaacatttcggggggtg | TRUE | 30.70 | 9.33 | 18.48 | 32.23 | 22.68 |
| 107 | 45783273 | 21 | −1 | ctcttggctctcggagccgca | FALSE | 107.28 | 44.44 | 37.83 | 71.88 | 65.36 |
| 108 | 45783251 | 21 | 1 | cctgcggctccgagagccaag | FALSE | 64.13 | 37.14 | 34.19 | 57.31 | 48.19 |
| 109 | 45783374 | 21 | 1 | gaaactaggaggcaaggaccg | FALSE | 15.29 | 7.19 | 25.40 | 16.57 | 16.11 |
| 110 | 45783472 | 21 | 1 | atgaggggccctggctgcggg | FALSE | 66.04 | 35.62 | 63.12 | 67.56 | 58.08 |
| 111 | 45783492 | 21 | 1 | gcaggatgctcttctccccaa | FALSE | 25.51 | 13.73 | 34.08 | 12.31 | 21.41 |
| 112 | 45783516 | 21 | 1 | ggtcctgctccagccgctggt | FALSE | 79.19 | 53.78 | 58.44 | 79.21 | 67.65 |
| 113 | 45783584 | 21 | 1 | ccggcttttcctgctatgaaa | TRUE | 114.69 | 80.57 | 112.83 | 128.55 | 109.16 |
| 114 | 45783666 | 21 | −1 | gcagtccgctttccccagagg | TRUE | 60.18 | 58.59 | 47.44 | 45.52 | 52.93 |
| 115 | 45783655 | 21 | 1 | aaagcggactgcctagaacca | FALSE | 83.49 | 29.31 | 29.64 | 74.09 | 54.13 |
| 116 | 45783834 | 21 | −1 | ttgctattgtctgtgcttttg | TRUE | 87.37 | 85.12 | 101.17 | 85.36 | 89.76 |
| 117 | 45783821 | 21 | 1 | acagacaatagcaagggcagc | TRUE | 36.68 | 36.14 | 42.16 | 52.36 | 41.83 |
| 118 | 45783956 | 21 | 1 | gaccagtcacatgctggggac | TRUE | 53.49 | 79.58 | 84.52 | 58.10 | 68.92 |
| 119 | 45783962 | 21 | 1 | tcacatgctggggacagggat | TRUE | 16.32 | 32.12 | 31.38 | 23.36 | 25.80 |
| 120 | 45784192 | 21 | 1 | atggctaggaggctgggggca | TRUE | 67.44 | 80.24 | 82.09 | 78.30 | 77.02 |
| 121 | 45784257 | 19 | −1 | ggcacagtggtgtgaagcc | TRUE | 100.96 | 107.71 | 94.81 | 92.64 | 99.03 |
| 122 | 45784350 | 21 | 1 | gtcaaggggagggagccagcc | TRUE | 88.94 | 97.88 | 86.05 | 78.71 | 87.89 |
| 123 | 45784387 | 21 | 1 | gctgccaccctggccttgccc | TRUE | 74.58 | 91.91 | 94.32 | 27.60 | 72.10 |
| 124 | 45784627 | 21 | −1 | tgagttctttccccagcacct | TRUE | 99.38 | 101.58 | 87.81 | 106.90 | 98.92 |
| 125 | 45784649 | 21 | −1 | gtggtagaggtaggactgtcc | TRUE | 86.07 | — | 51.37 | — | 68.72 |
| 126 | 45784636 | 21 | 1 | cctacctctaccactgacttg | TRUE | 111.67 | 112.66 | 120.50 | — | 114.94 |

In Table 1, “Coordinate” indicates the coordinate of the 5′ end of each sequences set forth in SEQ ID NOs: 4-126.

30 targeting sequences showed a reduction in DMPK expression of not less than 50% (SEQ ID NOs: 43, 44, 46, 62, 63, 66, 68, 70, 71, 72, 73, 80, 81, 82, 83, 85, 86, 88, 91, 96, 99, 100, 103, 105, 106, 108, 109, 111, 117 and 119), nine targeting sequences showed a reduction in DMPK expression of not less than 75% (SEQ ID NOs: 63, 70, 71, 83, 99, 105, 106, 109 and 111), and one targeting sequence showed a reduction in DMPK expression of not less than 80% (SEQ ID NO: 109).

Zones were identified and characterized based on the likelihood of the system described above of suppressing the expression of DMPK. In Zone 1 (FIG. 3: Chr19: GRCh38.p12; 45,777,342-45,778,884), we found that targeting sequences were ineffective in modulating the expression of DMPK. However, in Zone 2 (FIG. 3: GRCh38.p12; 45,778,884-45,783,985) targeting dSaCas9-KRAB was able to suppress DMPK expression. As expected, this region encompasses the DMPK promoter and transcription start site, suggesting that targeting this region has the largest effect on DMPK expression. Finally, Zone 3 (FIG. 3; Chr19: GRCh38.p12; 45,783,985-45,784,715) has less effect on DMPK expression and is more distant from the DMPK promoter region.

### Example 2 Adeno-Associated Virus (AAV) Production

### (1) Experimental Methods

Construction of plasmids for delivery and expression of dSaCas9-KRAB:gRNA and generation of AAV

pAAV-CMV was purchased from Takara (#6230) and EFS promoter sequence (SEQ ID NO: 204) and SV40 NLS-dSaCas9-NLS-KRAB (SEQ ID NO: 151) with an additional terminal stop codon [SEQ ID NO: 200 (DNA) and SEQ ID NO: 152 (protein)] were subcloned from pED162 (see Example 1). A bGlobin polyA sequence (SEQ ID NO: 201), U6 promoter sequence (SEQ ID No: 202), and SaCas9 gRNA scaffold sequence (SEQ ID NO: 150) were subcloned from pED0001 (SEQ ID NO: 203), thus replacing sequences encoding all functional components of pAAV-CMV (i.e. CMV promoter, beta-globin intron, MCS, and hGH polyA) between the ITRs. Finally, the EFS promoter was replaced with the CK8 promoter (SEQ ID NO: 187) by restriction cloning (XhoI and AgeI), resulting in plasmid pED148. The targeting sequence set forth in SEQ ID NO: 83, 70, 81, or 99 was cloned by digesting pED148 with BsaI, thus generating overhangs compatable with annealed synthetic oligos. Synthetic oligos were designed such that the forward primer had CACC(G) sequence at the 5′ end [5′CACC-(G)-targeting sequence-3′], and the reverse primer contained an additional AAAC sequence at the 5′ end [5′AAAC—reverse complement targeting sequence-(C)-3′]. An additional G was added to the beginning of the targeting sequence to enhance expression from the U6 promoter. The generated plasmids were named pED148-h695 (comprising the targeting sequence set forth in SEQ ID NO: 83), pED148-h245 (comprising the targeting sequence set forth in SEQ ID NO: 70), pED148-h257 (comprising the targeting sequence set forth in SEQ ID NO: 81), and pED148-h269 (comprising the targeting sequence set forth in SEQ ID NO: 99), respectively.

### Adeno-Associated Virus (AAV) Production

Adeno-associated virus serotype 9 (AAV9) particles were generated using 293T cells (ATCC #CRL-3216) seeded at a density of 0.86×10⁷ cells per Hyperflask (Corning #10030) and cultured in DMEM media (Sigma #D5796) supplemented with 10% FBS. Four days after seeding, media was changed to DMEM media supplemented with 2% FBS and 63 mM HEPES (Gibco #15630-080). The pRC9 plasmid was constructed as follows: AAV9 capsid sequence (see JP5054975B) was subcloned into a pRC2-mi342 vector (Takara #6230) replacing with that of AAV2 capsid sequence. Cells were transfected with 135 μg of the pRC9 plasmid, 121 μg of pHelper vector included in AAVpro® Helper Free System (Takara #6230) and 133 μg of one of pED148-h695, with 388 μl PEipro® in vitro DNA Transfection Reagent (Polyplus #115-010) per Hyperflask. After 3 days, 0.2% TritonX-100 was added to Hyperflask and cells were harvested.

After harvesting, its supernatant and cell lysate were clarified with cartridge filters (GE Healthcare #KGF-A-0506GG, KMP-HC9206GG). After the clarification, it was ultra filtrated with tangential flow filtration using the hollow fiber using the Xampler™ Ultrafiltration Cartridge, 750 kD (GE Healthcare #UFP-750-C-6MA). After reducing the volume, the sample was subjected to affinity chromatography (POROS™ CaptureSelect™ AAVX Affinity Resin (ThermoFisher Scientific #A36739)) for purifying AAV. Following the affinity chromatography step, the eluted sample was subjected to density gradient centrifugation for separating AAV from intermediate AAV particles. AAV particles separated with CsCl density gradient centrifugation, were subjected to buffer exchange with dialysis of phosphate buffered saline. After the buffer exchange, the AAV sample was concentrated using the Amicon® Ultra-4 Centrifugal Filter Unit (Merck millipore #UFC801024) and sterilized using the Millex-GV Syringe Filter Unit, 0.22 μm (Merck millipore #SLGV033RS). The AAV genome was purified with DNeasy Blood and Tissue Kit (QIAGEN #69506). The titer of purified AAV genome was measured using AAVpro® Titration Kit (for Real Time PCR) (Takara #6233). Resulting AAV denotes AAV9-695.

AAVs using pED148-h245, pED148-h257, or pED148-h269, were manufactured as described above and named AAV9-245, AAV9-257, and AAV9-269, respectively. Each of AAV9-695, AAV9-245, and AAV9-257 was manufactured twice and used for in vitro and in vivo experiments.

### (2) Results

Genome titer of the AAVs is shown in Table 2.

TABLE 2

| | Concentration | Lot |
|---|---|---|
| AAV name | (vg/mL) | No. |
| AAV9-695 | 2.8 × 10¹² | Lot 1 |
| AAV9-245 | 3.6 × 10¹² | Lot 1 |
| AAV9-257 | 4.5 × 10¹² | Lot 1 |
| AAV9-269 | 5.5 × 10¹² | Lot 1 |
| AAV9-695 | 3.6 × 10¹³ | Lot 2 |
| AAV9-245 | 3.7 × 10¹³ | Lot 2 |
| AAV9-257 | 4.6 × 10¹³ | Lot 2 |

### Example 3. In-Vitro Pharmacological Evaluation of Recombinant AAV9 Carrying the Base Sequence Encoding dSaCas9, Transcriptional Repressor and sgRNA on DMPK Gene Repression

### (1) Experimental Methods

### Cell Culture and AAV Infection

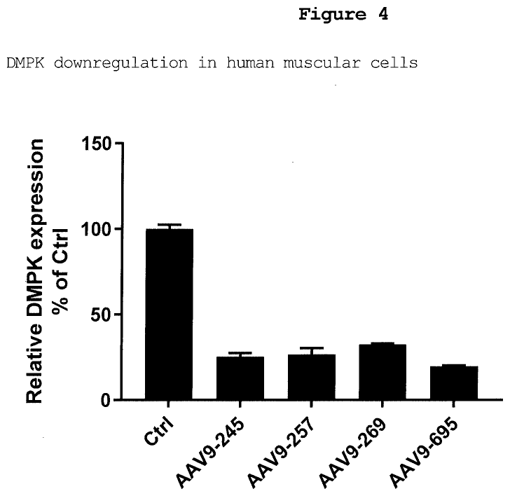

iCM cells were suspended in skeletal muscle cell growth medium kit (Promocell #C23060) (note: media was supplemented with 20% FBS, rather than 5% as directed by the kit, and 50 μg/ml Gentamicin S) and seeded into a Collagen type I-Coated 24 well plate (IWAKI #4820-010) at a density of 20,000 cells in 900 μl of medium per well. For AAV infection, 100 μl PBS with 0.001% Pluronic™ F-68 (GE healthcare #SH30594.01) containing 2.8, 3.6, 4.5, or 5.5×10¹² vg/ml of AAV9-695, AAV9-245, AAV9-257, or AAV9-269 were added to the medium and cultured and incubated at 37° C./5% CO₂ for 2 days. For control wells, 100 μl PBS with 0.001% Pluronic F-68 was added to the medium. The experiment was performed in tripricate. The media was replaced with differentiation media (DMEM media (Thermo Fisher #61965-026) supplemented with 10 μg/ml insulin (Sigma #19278)) and the cells were cultured for 4 days at 37° C. with 5% CO₂. After washing with 500 μl PBS, total RNA was extracted using RNeasy Plus Mini Kit (Qiagen #74134) according to the manufacturer's instruction. RNA from cells without AAV infection was set as control and shown as Ctrl in FIG. 4.

### Gene Expression Analysis

For Taqman qPCR, 80 ng of total RNA was converted to cDNA using SuperScript™ VILO™ cDNA Synthesis Kit (Thermo Fisher #11754250) in 20 μl reaction volume. The cDNA was diluted 160 fold with water and 2 μl was used for the qPCR. The qPCR was run in 5 μl final volume containing Taqman probes for DMPK (Thermo Fisher #Hs01094329 ml, FAM) or GAPDH (Thermo Fisher #Hs99999905_m1, FAM), and Taqman™ Gene Expression Master Mix (Thermo Fisher #4369016) with QuantStudio™ 12K Flex Real-Time PCR System (Thermo Fisher). The qPCR cycling condition was as follows: 95° C. for 10 minutes after 50° C. for 2 minutes followed by 45 cycles of 95° C. for 15 seconds and 60° C. for 1 minutes. The data were analyzed with QuantStudio™ 12K Flex software (Thermo Fisher). The expression values were analyzed with the standard curve for each gene and the expression level of DMPK gene was normalized to that of GAPDH gene.

### (2) Results

By applying AAV9-695, AAV9-245, AAV9-257, or AAV9-269 into iCM cells, DMPK mRNA downregulation was found, which suggests AAV9 carrying transgenes of dSaCas9, KRAB, and sgRNA comprising crRNA encoded by the targeting sequence set forth in SEQ ID NO: 83, 70, 81 or 99, has a pharmacological effect on DMPK downregulation in human muscular cells (FIG. 4).

### Example 4. Suppression of DMPK Gene Expression in DMSXL Mice

### (1) Experimental Methods

### Animal Treatment

AAV9-695, AAV9-245, or AAV9-257 was injected to DMSXL homo mice (termed DMSXL mice), transgenic mice carrying the human DM1 locus and very large expansions >1,000 CTG (PLoS Genet. 2012; 8(11):e1003043), intravenously (male n=2 and female n=2, in total n=4, respectively). Doses were as follows; 1.5×10¹³ vg/kg, 5×10¹³ vg/kg, 1.5×10¹⁴ vg/kg, and 5×10¹⁴ vg/kg for AAV9-695, AAV9-245, and AAV9-257, respectively. As a control, PBS containing 0.001% Pluronic F-68 (GE healthcare #SH30594.01) was injected. After 4 weeks, DMSXL mice were sacrificed and samples collected (tibialis anterior (TA), heart, and liver) from these mice. Gene expression analysis were performed on these samples as follows. Samples were stored in −80° C. freezing chamber until RNA extraction.

### RNA Extraction and Gene Expression Analysis

Tissue samples were homogenized using TissueLyser II (Qiagen) in 1 ml of ISOGEN (NIPPON GENE #319-90211). After centrifugal separation, 700 μl of supernatant was transferred to 1.5 ml tube containing 150 μl of chloroform (Wako #034-02603). After voltex and centrifuge, 187 μl of water layer was added to 150 μl of isopropanol (WAKO #166-04836) and mixed. The RNA extract was transferred to RNeasy spin columns of RNeasy® Plus Mini Kit (QIAGEN #74134) and further purified following manufacturer's protocol.

For Taqman qPCR, 700-1,000 ng of total RNA was converted to cDNA using SuperScript™ VILO™ cDNA Synthesis Kit (Thermo Fisher #11754250) in 20 μl reaction volume. The cDNA was diluted 20 fold with water and 3-4 μl was used for the qPCR. The qPCR was run in 10 μl final volume containing Taqman probes for DMPK (Thermo Fisher #Hs01094329 ml, FAM) or GAPDH (Thermo Fisher #Mm99999915_g1, FAM), and Taqman Gene Expression Master Mix (Thermo Fisher #4369016) with QuantStudio™ 12K Flex Real-Time PCR System (Thermo Fisher). The qPCR cycling condition was as follows: 95° C. for 10 minutes after 50° C. for 2 minutes followed by 40-45 cycles of 95° C. for seconds and 60° C. for 1 minutes. The data were analyzed with QuantStudio™ 12K Flex software (Thermo Fisher). The expression values were analyzed with the standard curve for each gene and the expression level of DMPK gene was normalized to that of GAPDH gene.

### (2) Results

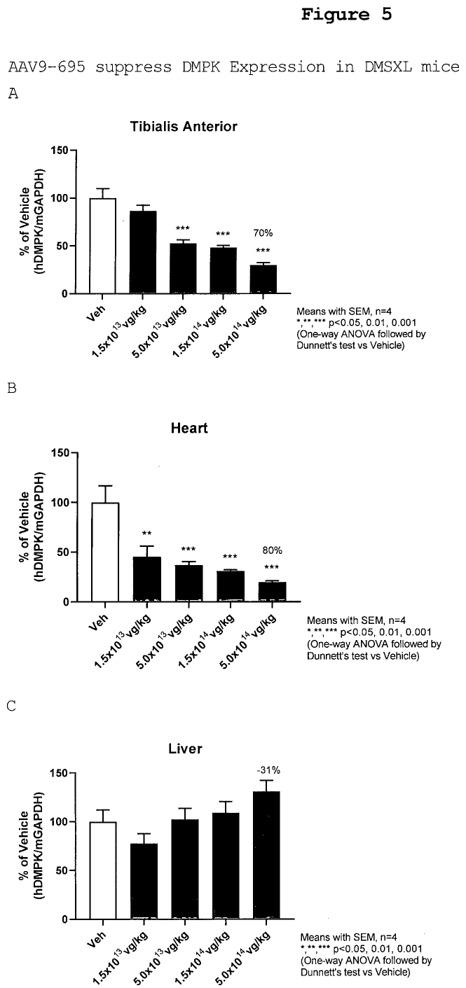

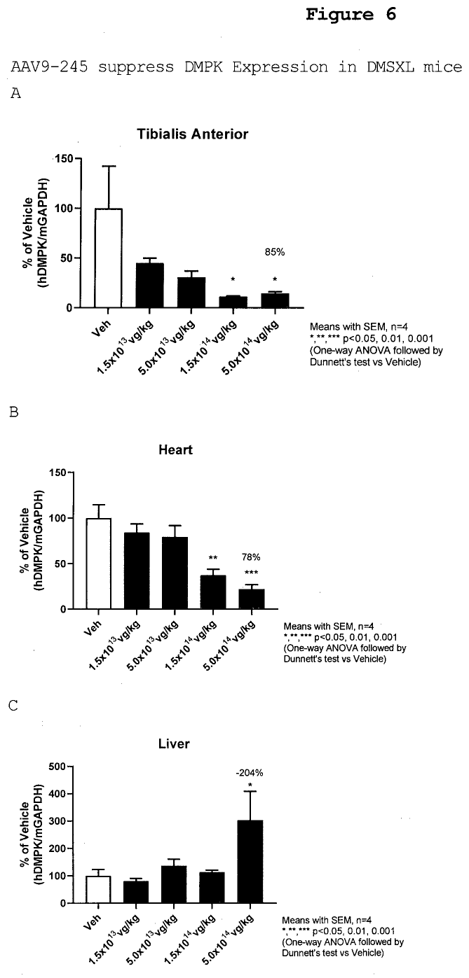

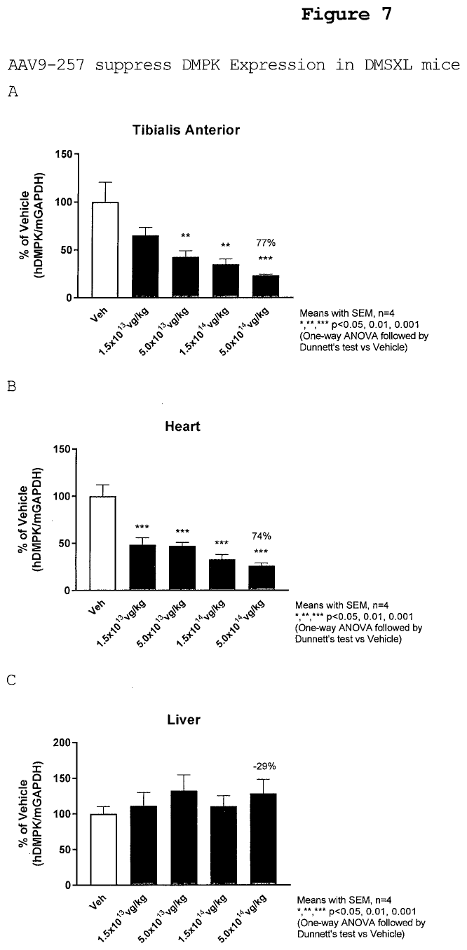

AAV9-695, AAV9-245, and AAV9-257 expressed each transgene in mice. DMPK mRNA downregulation was not found in liver but found in skeletal muscles and cardiac muscles, which suggests AAV9 carrying the transgene of dSaCas9, KRAB, and sgRNA comprising crRNA encoded by the targeting sequence set forth in SEQ ID NO: 83, 70 or 81 has a pharmacological effect on DMPK downregulation in DMSXL mice (FIGS. 5-7).

### Example 5. Improvement of RNA Foci Formation by AAV9-695 Administration to DMSXL Mice

### (1) Experimental Methods

### Fluorescence In Situ Hybridization: FISH

Administrations of AAV9-695 (5×10¹⁴ vg/kg) or vehicle (PBS containing 0.001% Pluronic F-68) as a control to DMSXL mice were conducted as described in Example 4. 4 weeks after administration, tibialis anterior (TA) muscles of DMSXL mice were excised and collected. After immediately embedded in Tissue-Tek® O.C.T. Compound (Sakura Finetek Japan, #4583), tissues were frozen in cold isopentane which is pre-chilled in liquid nitrogen and stored at −80° C.

10 μm of frozen sections of the tissue were prepared by a cryostat microtome and the thin sections were put on glass slides. The slides were air-dried and fixed with 4% paraformaldehyde at room temperature for 15 minutes and washed twice with PBS for 2 minutes and stored at 4° C.

After incubation in PBS containing 2% acetone for 5 minutes at room temperature, the slides were incubated in 2×saline sodium citrate buffer (SSC) (300 mM NaCl and 30 mM Sodium Citrate) containing 30% formamide for 10 minutes at room temperature. The slides were incubated in probe solution (0.02% bovine serum albumin (SIGMA #A7030-100G), 0.066 mg/ml yeast tRNA (Thermo Fisher #15401-011), 2 mM ribonucleoside vanadyl complex (SIGMA #R3380-5ML), and 1 ng/μl Cy3-(CAG)5-2′-OMe probe (y_C(M)A(M)G(M)C(M)A(M)G(M)C(M)A(M)G(M)C(M)A(M)G(M)C(M)A(M)G(M), y means Cy3 and N(M) means 2′-OMe RNA. This probe was synthesized by GeneDesign, Inc., Japan) in 2×SSC containing 30% formamide) for 2 hours at 37° C. After hybridization, the probe solution was removed and the slides were incubated in 2×SSC containing 30% formamide for 30 minutes at 50° C. The slides were washed once with 1×SSC and incubated in 1×SSC for 30 minutes at room temperature. The slides were washed three times with PBS for 10 minutes and ProLong™ Diamond Antifade Mountant with DAPI (Thermo Fisher #P36971) was added to the slide. The slides were covered with cover slips and stored at 4° C.

Formation of RNA foci were observed using confocal laser microscope LSM700 (ZEISS).

### (2) Results

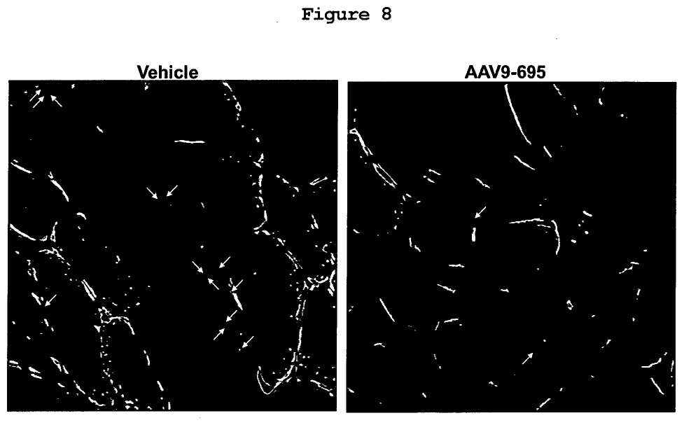

Typical images of TA muscle section of DMSXL mice administered with the vehicle or AAV9-695 are shown in FIG. 8. Arrows indicate RNA foci (RNA foci was defined as a clearly detectable red dot localizing in nucleus colored blue).

The numbers of RNA foci observed in TA muscles of AAV9-695-administered DMSXL mice were lower than in TA muscles of vehicle-administered DMSXL mice, suggesting that AAV9-695 administration improved RNA foci formations in DMSXL mice.

### Example 6. Suppression of DMPK Gene Expression in hDMPK sgRNA-Expressing iDM Cells

### (1) Experimental Methods

### Lentivirus Generation

Lenti-X™ 293T Cells (Takara #632180) were seeded at 5×10⁶ cells/dish in collagen type I-coated dish 100 mm (IWAKI #4020-010) in 10 ml DMEM (Thermo Fisher #10569-010) supplemented with 10% FBS and MEM Non-Essential Amino Acids Solution (Thermo. Fisher #11140050)) and incubated at 37° C./5% CO₂ overnight. The next day Lipofectaminen™ 3000 Transfection Reagent (Thermo Fisher #L3000008) was set up according to manufacturer's protocol with 7 μg of Lentiviral High Titer Packaging Mix (Takara #6194) and 5.5 μg of transfer plasmid pED162 containing sequence encoding dSaCas9-KRAB and indicated targeting sequence set forth in SEQ ID NO: 1 or 83 (Example 1). Plasmids are named as described in Table 3. 10 ml of media containing lentivirus was harvested 48 hours following transfection by passing media supernatant through a 0.45 μm filter. To concentrate virus solution, ¼ volume of PEG-It™ Virus Precipitation Solution (SBI #LV810A-1) was added and incubated overnight at 4° C. The supernatant was centrifuged at 1,500×g for 30 minutes. After discarding the supernatant, 200 μl of DMEM was added to the tube and virus solution was resuspended gently and stored at −80° C.

TABLE 3

| plasmid | SEQ ID NO |
|---|---|
| pED162-C1 | 1 |
| pED162-695 | 83 |

Lentivirus titers ranged from 5×10¹⁰ to 7×10¹⁰ particles/ml, measured by using NucleoSpin® RNA Virus (MACHEREY-NAGEL #740956.250) and Lenti-X™ qRT-PCR Titration Kit (Clontech #631235).

### Transduction of iDM Cells

iDM cells were seeded at 50,000 cells/well in collagen type I-coated 12 well plate (IWAKI #4815-010) in 1 ml medium containing growth medium [PromoCell Skeletal Muscle Cell Growth Medium Kit; part number: C-23060 (note: media was supplemented with 20% FBS, rather than 5% as directed by kit, and 50 μg/ml Gentamicin S)] and incubated at 37° C./5% CO₂ overnight. The next day, the medium was replaced with 1 ml growth medium supplemented with 5 μg/ml Polybrene (Sigma, #TR-1003-G) and 0.03 ml lentivirus supernatant (see above) corresponding to each sgRNA comprising crRNA encoded by individual targeting sequences (SEQ ID No: 1 or 83) fused with tracrRNA was added to each well. Cells were incubated with lentivirus for 48 hours before viral media was removed and replaced with selection media [growth media supplemented with 10 μg/ml Blasticidin (Nacalai #03759-71)]. Following 24 hours of incubation in selection media one third of cells were passed into new wells with growth media. After allowing cells to seed for 72 hours, growth media were replaced with selection media. Following 48 hours of culture in selection media, cells were harvested and stocked.

### Transduction of iCM Cells

iCM cells were seeded at 50,000 cells/well in collagen type I-coated 6 well plate (IWAKI #4810-010) in 2 ml medium containing growth medium [PromoCell Skeletal Muscle Cell Growth Medium Kit; part number: C-23060 (note: media was supplemented with 20% FBS, rather than 5% as directed by kit, and 50 μg/ml Gentamicin S)] and incubated at 37° C./5% CO₂ overnight. The next day, the medium was replaced with 2 ml is growth medium supplemented with 5 μg/ml Polybrene (Sigma #TR-1003-G) and 2×10⁹ vg lentivirus supernatant (see above) corresponding to Control sgRNA comprising crRNA encoded by individual targeting sequence (SEQ ID No: 1) fused with tracrRNA was added to each well. Cells were incubated with lentivirus for 48 hours before viral media was removed and replaced with selection media [growth media supplemented with 10 μg/ml Blasticidin (Nacalai #03759-71)]. Following 24 hours of incubation in selection media, two third of cells were passed into collagen type I-coated dish 100 mm (iwaki #4020-010) with growth media. After allowing cells to seed for 72 hours, growth media were replaced with selection media. Following 48 hours of culture in selection media, cells were harvested and stocked.

Cell Culture, RNA Extraction, and cDNA Preparation

iDM cells expressing dSaCas9 and hDMPK sgRNA comprising crRNA encoded by the targeting sequence set forth in SEQ ID: 83, iDM cells expressing dSaCas9 and control sgRNA comprising crRNA encoded by the targeting sequence set forth in SEQ ID NO: 1, and iCM cells expressing dSaCas9 and control sgRNA comprising crRNA encoded by the targeting sequence set forth in SEQ ID NO: 1, termed iDM-695 cells (iDM_695), iDM-Ctrl cells (iDM_Ctrl), and iCM-Ctrl cells (iDM_Ctrl) respectively, were seeded into a collagen type I-coated 24 well plate (IWAKI #4820-010) at a density of 25,000 cells in 500 μl or 50,000 cells per well in in 1 ml of skeletal muscle cell growth medium kit (Promocell #C23060) supplemented with 20% of non-heat inactivated FBS and incubated at 37° C./5% CO₂ for 2 days (seeded at 50,000 cells/well) or 3 days (seeded at 25,000 cells/well).

After washing with 200 μL PBS, total RNA was extracted using RNeasy Mini Kit (Qiagen #74106) according to the manufacturer's instruction.

500 ng of total RNA was converted to cDNA using SuperScript™ VILO™ cDNA Synthesis Kit (Thermo Fisher #11754-250) according to the manufacturer's instruction. The cDNA was stored at −20° C.

### Gene Expression Analysis

The cDNA was diluted 100-fold with water and 2 μl was used for the qPCR. The qPCR was run in 10 μl final volume containing Taqman probes for DMPK (Thermo Fisher #Hs01094329 ml, FAM) or for GAPDH (Thermo Fisher #Hs99999905 ml, FAM), and Taqman Gene Expression Master Mix (Thermo Fisher #4369016) with ViiA7 Real Time PCR System (Thermo Fisher). The qPCR condition was as follows: pre-heated with 50° C. for 2 minutes and 95° C. for 10 minutes followed by 45 cycles of 95° C. for 15 seconds and 60° C. for 1 minutes. The expression values were analyzed with the standard curve for each gene and the expression level of DMPK gene was normalized to that of GAPDH gene.

### (2) Results

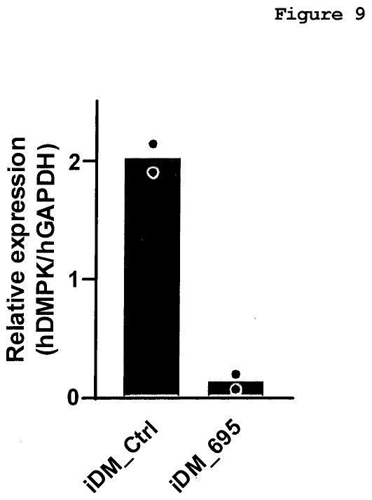

Expressions of DMPK gene in iDM-695 cells and those in -iDM-Ctrl cells are shown in FIG. 9.

DMPK gene expression was suppressed in hDMPK sgRNA-expressing iDM cells.

### Example 7. Improvement of RNA Foci Formation in hDMPK sgRNA-Expressing iDM Cells

### (1) Experimental Methods

### Fluorescence In Situ Hybridization: FISH

iDM-695 cells, iDM-Ctrl cells, and iCM-Ctrl cells, which were constructed in Example 6, were seeded quadruplicate into a collagen-coated 96 well plate (Thermo Fisher Scientific #152038) at a density of 2,500 cells or 5,000 cells per well in skeletal muscle cell growth medium kit (Promocell #C23060) supplemented with 20% of non-heat inactivated FBS and incubated at 37° C./5% CO₂ for 2 days (seeded at 5,000 cells/well) or 3 days (seeded at 2,500 cells/well).

The cells were washed twice with phosphate buffered saline (PBS), fixed with 4% paraformaldehyde at room temperature for 15 minutes, washed twice with PBS, and stored at 4° C.

After incubation in PBS containing 0.2% Triton X-100 for 10 minutes at room temperature, the cells were washed and incubated in 2×SSC containing 40% formamide for 10 minutes at room temperature. 50 μl of probe solution (0.02% bovine serum albumin (SIGMA #A7030-100G), 0.066 mg/ml yeast tRNA (Thermo Fisher Scientific #15401-011), 2 mM ribonucleoside vanadyl complex (SIGMA #R3380-5ML), and 0.1 ng/μl Cy3-(CAG)5-LNA probe (y_5(L)A(L)G(L)cagcagcag5(L)A(L)G(L), y means Cy3, 5(L) means LNA-mC, N(L) means LNA, and lower case means DNA. This probe was synthesized by GeneDesign, Inc.) in 2×SSC containing 40% formamide) was added to each well and the cells were incubated for 2 hours at 37° C. After hybridization, the probe solution was removed and the cells were incubated in 2×SSC containing 40% formamide for 30 minutes at 37° C. The cells were washed once with 1×SSC and incubated in 1×SSC for 30 minutes at room temperature. 50 μl of PBS containing 2 μg/ml DAPI (Dojindo #340-07971) was added to each well and the cells were incubated for 30 minutes at room temperature. The cells were washed twice with PBS for 5 minutes at room temperature and stored at 4° C.

Formation of RNA foci was detected and analyzed using IN Cell Analyzer 6000 (GE healthcare). The images of 9 points in each well were captured and the number of RNA foci positive nuclei and the total number of nuclei in each image were counted. The ratio of foci positive nuclei in each well was analyzed and averages were calculated.

### (2) Results

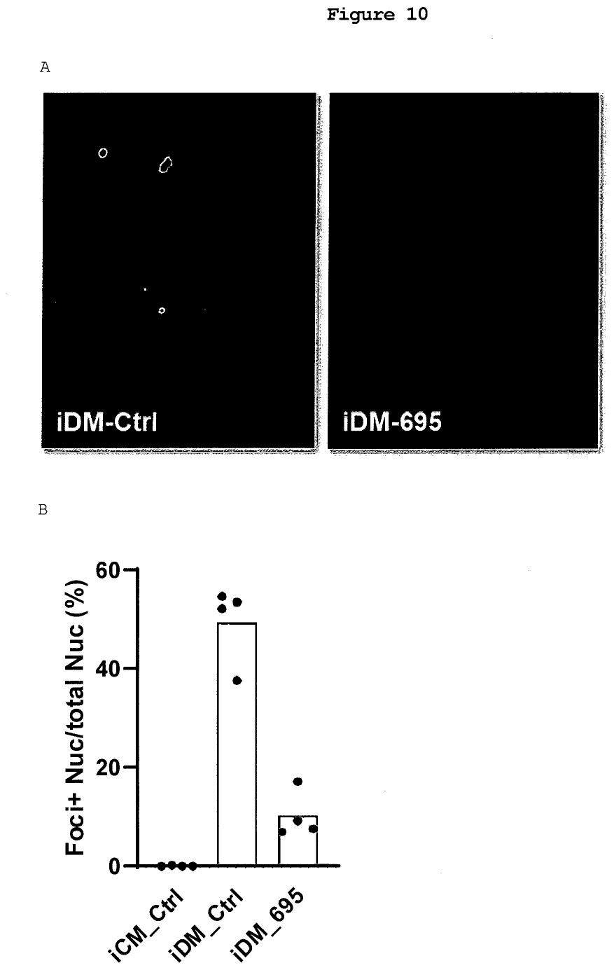

Typical images of iDM-695 cells and iDM-Ctrl cells are shown in FIG. 10A.

The ratios of RNA foci positive nuclei in each cell are shown in FIG. 10B.

The ratios of RNA foci positive nuclei in iDM-695 cells were lower than those in iDM-Ctrl cells.

### Example 8. Improvement of Splicing Defects in hDMPK sgRNA-Expressing iDM Cells

### (1) Experimental Methods

### Splicing Analysis

Preparations of cDNAs from iDM-695 cells, iDM-Ctrl cells, and iCM-695 cells were described in Example 6.

PCR was conducted using PrimeSTAR® GXL DNA Polymerase (TaKaRa #R050A) according to the manufacturer's instruction. The cDNA was diluted 10-fold with water and 1 μl was used. The PCR primers used were as follows:

TABLE 4

| Target | Forward 5′→3′ | Reverse 5′→3′ |
|---|---|---|
| DMD exon 78 | TTAGAGGAGGTGATGGAGCA | GATACTAAGGACTCCATCGC |
| | (SEQ ID NO: 192) | (SEQ ID NO: 193) |
| MBNL1 exon 7 | GCTGCCCAATACCAGGTCAAC | TGGTGGGAGAAATGCTGTATGC |
| | (SEQ ID NO: 194) | (SEQ ID NO: 195) |
| KIF13A exon 21 | ACCTGTGCAGCATTCAGGGACAC | CTCGTCGTTTAATGAGTGCATCTG |
| | (SEQ ID NO: 196) | (SEQ ID NO: 197) |
| TNNT2 exon 5 | ATAGAAGAGGTGGTGGAAGAGTAC | GTCTCAGCCTCTGCTTCAGCATCC |
| | (SEQ ID NO: 198) | (SEQ ID NO: 199) |

The PCR cycle condition was as follows: 35 cycles of 98° C. for 10 seconds, 60° C. for 15 seconds, and 68° C. for 30 seconds followed by 72° C. for 7 minutes.

The PCR products were loaded on Agilent DNA1000 Kit (Agilent #5067-1504), electrophoresed, and analyzed using Agilent 2100 BioAnalyzer system according to the manufacturer's instruction.

AUCs of the peaks of normally and abnormally spliced products were measured, and the ratios of the normally spliced products in each cell were calculated.

### (2) Results

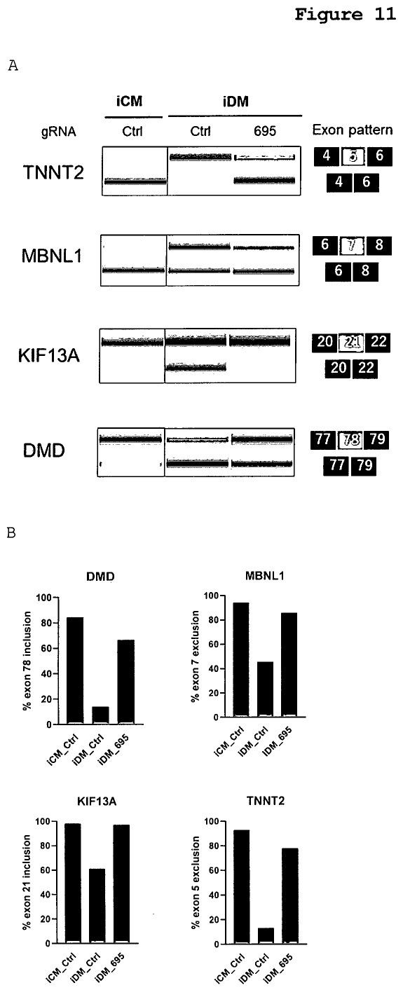

Gel images and exon patterns of each gene, i.e. DMD, MBNL1, KIF13A, and TNNT2, are shown in FIG. 11A.

The ratios of normally spliced products, which are more abundant in iCM cells and less in iDM cells, in each cell are shown in FIG. 11B.

Splicing defects of all the genes tested were improved in iDM-695 cells.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

### INDUSTRIAL APPLICABILITY

According to the present invention, expression of DMPK gene can be suppressed in the cells derived from DM1 patients and the DM1 model mice. Therefore, the present invention is expected to be extremely useful for the treatment and/or prevention of DM1.

1: A polynucleotide comprising the following base sequences:
(a) a base sequence encoding a fusion protein of a nuclease-deficient CRISPR effector protein and a transcriptional repressor, and
(b) a base sequence encoding a guide RNA targeting a continuous region of 18 to 24 nucleotides in length in a region set forth in SEQ ID NO: 127, SEQ ID NO: 46, SEQ ID NO: 128, SEQ ID NO: 129, SEQ ID NO: 130, SEQ ID NO: 131, SEQ ID NO: 132, SEQ ID NO: 88, SEQ ID NO: 91, SEQ ID NO: 133, SEQ ID NO: 137, SEQ ID NO: 117, or SEQ ID NO: 119 in the expression regulatory region of human DMPK gene.

2: The polynucleotide according to claim 1, comprising the following base sequences:
(a) a base sequence encoding a fusion protein of a nuclease-deficient CRISPR effector protein and a transcriptional repressor, and
(b) a base sequence encoding a guide RNA targeting a continuous region of 18 to 24 nucleotides in length in a region set forth in SEQ ID NO: 127, SEQ ID NO: 46, SEQ ID NO: 128, SEQ ID NO: 66, SEQ ID NO: 68, SEQ ID NO: 130, SEQ ID NO: 131, SEQ ID NO: 132, SEQ ID NO: 88, SEQ ID NO: 91, SEQ ID NO: 96, SEQ ID NO: 99, SEQ ID NO: 100, SEQ ID NO: 134, SEQ ID NO: 108, SEQ ID NO: 109, SEQ ID NO: 111, SEQ ID NO: 117, or SEQ ID NO: 119 in the expression regulatory region of human DMPK gene.

3: The polynucleotide according to claim 1, comprising the following base sequences:
(a) a base sequence encoding a fusion protein of a nuclease-deficient CRISPR effector protein and a transcriptional repressor, and
(b) a base sequence encoding a guide RNA targeting a continuous region of 18 to 24 nucleotides in length in a region set forth in SEQ ID NO: 63, SEQ ID NO: 136, SEQ ID NO: 83, SEQ ID NO: 99, SEQ ID NO: 135, SEQ ID NO: 109, or SEQ ID NO: 111 in the expression regulatory region of human DMPK gene.

4: The polynucleotide according to claim 1, wherein the base sequence encoding the guide RNA comprises the base sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117, or SEQ ID NO: 119, or the base sequence set forth in SEQ ID NO: 43, SEQ ID NO: 44, SEQ ID NO: 46, SEQ ID NO: 62, SEQ ID NO: 63, SEQ ID NO: 66, SEQ ID NO: 68, SEQ ID NO: 70, SEQ ID NO: 71, SEQ ID NO: 72, SEQ ID NO: 73, SEQ ID NO: 80, SEQ ID NO: 81, SEQ ID NO: 82, SEQ ID NO: 83, SEQ ID NO: 85, SEQ ID NO: 86, SEQ ID NO: 88, SEQ ID NO: 91, SEQ ID NO: 96, SEQ ID NO: 99, SEQ ID NO: 100, SEQ ID NO: 103, SEQ ID NO: 105, SEQ ID NO: 106, SEQ ID NO: 108, SEQ ID NO: 109, SEQ ID NO: 111, SEQ ID NO: 117, or SEQ ID NO: 119 in which 1 to 3 bases are deleted, substituted, inserted, and/or added.

5: The polynucleotide according to claim 1 comprising at least two base sequences encoding the guide RNAs, wherein the at least two base sequences are different.

6: The polynucleotide according to claim 1, wherein the transcriptional repressor is selected from the group KRAB, MeCP2, SIN3A, HDT1, MBD2B, NIPP1, and HP1A.

7: The polynucleotide according to claim 6, wherein the transcriptional repressor is KRAB.

8: The polynucleotide according to claim 1, wherein the nuclease-deficient CRISPR effector protein is dCas9.

9: The polynucleotide according to claim 8, wherein the dCas9 is derived from Staphylococcus aureus.

10: The polynucleotide according to claim 1, further comprising a promoter sequence for the base sequence encoding the guide RNA and/or a promoter sequence for the base sequence encoding the fusion protein of the nuclease-deficient CRISPR effector protein and the transcriptional repressor.

11: The polynucleotide according to claim 10, wherein the promoter sequence for the base sequence encoding the guide RNA is selected from the group U6 promoter, SNR6 promoter, SNR52 promoter, SCR1 promoter, RPR1 promoter, U3 promoter, and H1 promoter.

12: The polynucleotide according to claim 11, wherein the promoter sequence for the base sequence encoding the guide RNA is U6 promoter.

13: The polynucleotide according to claim 10, wherein the promoter sequence for the base sequence encoding the fusion protein of the nuclease-deficient CRISPR effector protein and the transcriptional repressor is a ubiquitous promoter or a muscle specific promoter.

14: The polynucleotide according to claim 13, wherein the ubiquitous promoter is selected from the group EFS promoter, CMV promoter and CAG promoter.

15: The polynucleotide according to claim 13, wherein the muscle specific promoter is selected from the group CK8 promoter, myosin heavy chain kinase (MHCK) promoter, muscle creatine kinase (MCK) promoter, synthetic C5-12 (Syn) promoter, and Des promoter.

16: The polynucleotide according to claim 15, wherein the muscle specific promoter is CK8 promoter.

17: The polynucleotide according to claim 10,
wherein the base sequence encoding the guide RNA comprises the base sequence set forth in SEQ ID NO: 70, SEQ ID NO: 81, SEQ ID NO: 83, or SEQ ID NO: 99, or the base sequence set forth in SEQ ID NO: 70, SEQ ID NO: 81, SEQ ID NO: 83, or SEQ ID NO: 99 in which 1 to 3 bases are deleted, substituted, inserted, and/or added,
the transcriptional repressor is KRAB,
the nuclease-deficient CRISPR effector protein is dCas9 derived from Staphylococcus aureus,
the promoter sequence for the base sequence encoding the guide RNA is U6 promoter, and
the promoter sequence for the base sequence encoding the fusion protein of the nuclease-deficient CRISPR effector protein and the transcriptional repressor is CK8 promoter.

18: The polynucleotide according to claim 17,
wherein the base sequence encoding the guide RNA comprises the base sequence set forth in SEQ ID NO: 83, or the base sequence set forth in SEQ ID NO: 83 in which 1 to 3 bases are deleted, substituted, inserted, and/or added.

19: A vector comprising a polynucleotide according to claim 1.

20: The vector according to claim 19, wherein the vector is a plasmid vector or a viral vector.

21: The vector according to claim 20, wherein the viral vector is selected from the group adeno-associated virus (AAV) vector, adenovirus vector, and lentivirus vector.

22: The vector according to claim 21, wherein the AAV vector is selected from the group AAV1, AAV2, AAV6, AAV7, AAV8, AAV9, Anc80, AAV₅₈₇MTP, AAV₅₈₈MTP, AAV-B 1, AAVM41, and AAVrh74.

23-25. (canceled)

26: A method for treating or preventing myotonic dystrophy type 1, comprising administering a polynucleotide according to claim 1, to a subject in need thereof.


SEQUENCE LISTING (204 sequences)

SEQ ID NO: 1 - DNA, 20 bp, Artificial Sequence
  control non-targeting targeting sequence
acggaggctaagcgtcgcaa

SEQ ID NO: 2 - DNA, 20 bp, Artificial Sequence
  control non-targeting targeting sequence
cgcttccgcggcccgttcaa

SEQ ID NO: 3 - DNA, 20 bp, Artificial Sequence
  control non-targeting targeting sequence
gtaggcgcgccgctctctac

SEQ ID NO: 4 - DNA, 21 bp, Homo sapiens
cccttcttctttggcctcgac

SEQ ID NO: 5 - DNA, 21 bp, Homo sapiens
cccagtcgaggccaaagaaga

SEQ ID NO: 6 - DNA, 21 bp, Homo sapiens
cccccggagacacggctgggc

SEQ ID NO: 7 - DNA, 21 bp, Homo sapiens
ctccgggggacacagcaaccg

SEQ ID NO: 8 - DNA, 21 bp, Homo sapiens
ctgggacaggcagctacgggc

SEQ ID NO: 9 - DNA, 21 bp, Homo sapiens
aggcccaccgcccacagcctg

SEQ ID NO: 10 - DNA, 21 bp, Homo sapiens
gatctcgggggacaggtagtc

SEQ ID NO: 11 - DNA, 21 bp, Homo sapiens
gcccagagctggtgggcccag

SEQ ID NO: 12 - DNA, 21 bp, Homo sapiens
gatgtggccacagcggtccag

SEQ ID NO: 13 - DNA, 21 bp, Homo sapiens
cagcggtccagcaggatgttg

SEQ ID NO: 14 - DNA, 21 bp, Homo sapiens
gatagcaagcttgttccctgg

SEQ ID NO: 15 - DNA, 21 bp, Homo sapiens
gtagaagcgcgccatctcggc

SEQ ID NO: 16 - DNA, 21 bp, Homo sapiens
actgctgagcaagtttgggga

SEQ ID NO: 17 - DNA, 21 bp, Homo sapiens
ccccttctcagtacctggtca

SEQ ID NO: 18 - DNA, 21 bp, Homo sapiens
ctccatgaccaggtactgaga

SEQ ID NO: 19 - DNA, 21 bp, Homo sapiens
ggtactgagaaggggttcgtc

SEQ ID NO: 20 - DNA, 21 bp, Homo sapiens
tggggctgcaggtctctgccc

SEQ ID NO: 21 - DNA, 21 bp, Homo sapiens
tcccagaacatctcaggggga

SEQ ID NO: 22 - DNA, 21 bp, Homo sapiens
tctttcccagaacatctcagg

SEQ ID NO: 23 - DNA, 21 bp, Homo sapiens
ctgggacctattctctgggtg

SEQ ID NO: 24 - DNA, 21 bp, Homo sapiens
ggagtgtctgggacctattct

SEQ ID NO: 25 - DNA, 21 bp, Homo sapiens
aacggggccactgcctaggga

SEQ ID NO: 26 - DNA, 20 bp, Homo sapiens
tccctaggcagtggccccgt

SEQ ID NO: 27 - DNA, 20 bp, Homo sapiens
ggggggtctcttacctggaa

SEQ ID NO: 28 - DNA, 21 bp, Homo sapiens
ggggccttccctatctgagac

SEQ ID NO: 29 - DNA, 21 bp, Homo sapiens
tgcggtaagagggtgtgtcag

SEQ ID NO: 30 - DNA, 21 bp, Homo sapiens
gggaaagtctgtgtgcggtaa

SEQ ID NO: 31 - DNA, 21 bp, Homo sapiens
gggagaggaagctctaggatt

SEQ ID NO: 32 - DNA, 21 bp, Homo sapiens
ggaggtggggagaggaagctc

SEQ ID NO: 33 - DNA, 21 bp, Homo sapiens
actacctggtgagctccgggc

SEQ ID NO: 34 - DNA, 21 bp, Homo sapiens
gttggtgaatggggaccggcg

SEQ ID NO: 35 - DNA, 21 bp, Homo sapiens
cgtgaggagagggacgtgttg

SEQ ID NO: 36 - DNA, 21 bp, Homo sapiens
cctgaggtcgagatagtgaga

SEQ ID NO: 37 - DNA, 21 bp, Homo sapiens
tgggtgcagaagcgggtggag

SEQ ID NO: 38 - DNA, 21 bp, Homo sapiens
acggctgggtgcagaagcggg

SEQ ID NO: 39 - DNA, 21 bp, Homo sapiens
ggggcggggccttagaaattg

SEQ ID NO: 40 - DNA, 21 bp, Homo sapiens
gactcgtggtgggcggggcat

SEQ ID NO: 41 - DNA, 21 bp, Homo sapiens
aacccctatgccccgcccacc

SEQ ID NO: 42 - DNA, 21 bp, Homo sapiens
cgccccgcgtccgatcacctt

SEQ ID NO: 43 - DNA, 19 bp, Homo sapiens
gtggtgggggaaaagaacc

SEQ ID NO: 44 - DNA, 21 bp, Homo sapiens
agagagaaggggagacagaca

SEQ ID NO: 45 - DNA, 21 bp, Homo sapiens
tccaagggtgtgcaggatggt

SEQ ID NO: 46 - DNA, 21 bp, Homo sapiens
gggtgtgcaggatggttaggg

SEQ ID NO: 47 - DNA, 21 bp, Homo sapiens
aggatggttagggtggggtaa

SEQ ID NO: 48 - DNA, 21 bp, Homo sapiens
tttccttctccccttgttctt

SEQ ID NO: 49 - DNA, 21 bp, Homo sapiens
tcagggtggaaggtggaagaa

SEQ ID NO: 50 - DNA, 20 bp, Homo sapiens
cctgaactgtcacctggagt

SEQ ID NO: 51 - DNA, 20 bp, Homo sapiens
cctgaactgtcacctggagt

SEQ ID NO: 52 - DNA, 21 bp, Homo sapiens
ggctgcacctgaactgtcacc

SEQ ID NO: 53 - DNA, 21 bp, Homo sapiens
ggacattctacatgagaacgt

SEQ ID NO: 54 - DNA, 21 bp, Homo sapiens
ccccacccccacgttctcatg

SEQ ID NO: 55 - DNA, 19 bp, Homo sapiens
cgttctcatgtagaatgtc

SEQ ID NO: 56 - DNA, 21 bp, Homo sapiens
tcctgggtaacggcccagacg

SEQ ID NO: 57 - DNA, 21 bp, Homo sapiens
tggttcaagaaccacctgcat

SEQ ID NO: 58 - DNA, 21 bp, Homo sapiens
tgaaccacactttggaaaacc

SEQ ID NO: 59 - DNA, 21 bp, Homo sapiens
cctgattgtcatctgtaaata

SEQ ID NO: 60 - DNA, 21 bp, Homo sapiens
agaagtcaaggtcctatgact

SEQ ID NO: 61 - DNA, 21 bp, Homo sapiens
gacagggaggcctggactccc

SEQ ID NO: 62 - DNA, 21 bp, Homo sapiens
aagtggaggcaggatggagag

SEQ ID NO: 63 - DNA, 21 bp, Homo sapiens
ggaggcaggatggagagaggg

SEQ ID NO: 64 - DNA, 21 bp, Homo sapiens
gcctggggcgggggagggggc

SEQ ID NO: 65 - DNA, 21 bp, Homo sapiens
agatgcagctcgggccacaaa

SEQ ID NO: 66 - DNA, 21 bp, Homo sapiens
gcagggtaaactgagaccggg

SEQ ID NO: 67 - DNA, 21 bp, Homo sapiens
gggagaaggtttttccagagg

SEQ ID NO: 68 - DNA, 21 bp, Homo sapiens
gacatatgagggccagagggg

SEQ ID NO: 69 - DNA, 21 bp, Homo sapiens
gggccagaggggcagggtgct

SEQ ID NO: 70 - DNA, 21 bp, Homo sapiens
agaaaccagtgaccagtgagc

SEQ ID NO: 71 - DNA, 21 bp, Homo sapiens
gcctggggccagggctggaag

SEQ ID NO: 72 - DNA, 21 bp, Homo sapiens
tcagccaccattcccgccacc

SEQ ID NO: 73 - DNA, 21 bp, Homo sapiens
gtctcggttcctatgagccgt

SEQ ID NO: 74 - DNA, 21 bp, Homo sapiens
cgagactttggggctgggggt

SEQ ID NO: 75 - DNA, 21 bp, Homo sapiens
actttggggctgggggtgggg

SEQ ID NO: 76 - DNA, 21 bp, Homo sapiens
gagggacaactttgggttctt

SEQ ID NO: 77 - DNA, 21 bp, Homo sapiens
aagaacccaaagttgtccctc

SEQ ID NO: 78 - DNA, 21 bp, Homo sapiens
tggttctcccacagggcccgc

SEQ ID NO: 79 - DNA, 21 bp, Homo sapiens
catagagcccacttttggggg

SEQ ID NO: 80 - DNA, 21 bp, Homo sapiens
tgtgactcagagccatggctt

SEQ ID NO: 81 - DNA, 21 bp, Homo sapiens
tgtgactcctaagccatggct

SEQ ID NO: 82 - DNA, 19 bp, Homo sapiens
tgactcagagccatggctt

SEQ ID NO: 83 - DNA, 21 bp, Homo sapiens
cagagtaaggtcagcagaggc

SEQ ID NO: 84 - DNA, 21 bp, Homo sapiens
gctgaccttactctgcccctc

SEQ ID NO: 85 - DNA, 19 bp, Homo sapiens
gggctcctgcagatggggt

SEQ ID NO: 86 - DNA, 21 bp, Homo sapiens
tggccgacttcttgcagtggg

SEQ ID NO: 87 - DNA, 21 bp, Homo sapiens
tacgtggccgacttcttgcag

SEQ ID NO: 88 - DNA, 21 bp, Homo sapiens
ggggctccagccccaggaagc

SEQ ID NO: 89 - DNA, 21 bp, Homo sapiens
cccctccccgggccgggggct

SEQ ID NO: 90 - DNA, 21 bp, Homo sapiens
ggcgggaggggggctggacca

SEQ ID NO: 91 - DNA, 21 bp, Homo sapiens
attccccctactctagcactg

SEQ ID NO: 92 - DNA, 21 bp, Homo sapiens
ctctgtgctctacctttttat

SEQ ID NO: 93 - DNA, 21 bp, Homo sapiens
ggagcaagggggtggttgcta

SEQ ID NO: 94 - DNA, 21 bp, Homo sapiens
acagaggaagccacaggtgtg

SEQ ID NO: 95 - DNA, 19 bp, Homo sapiens
gggaggccagggcagcttc

SEQ ID NO: 96 - DNA, 21 bp, Homo sapiens
agcttcttgggtgactcagag

SEQ ID NO: 97 - DNA, 21 bp, Homo sapiens
tgtccgagtcggaatccatct

SEQ ID NO: 98 - DNA, 21 bp, Homo sapiens
gctaaatttaactgtccgagt

SEQ ID NO: 99 - DNA, 21 bp, Homo sapiens
ctgagggctaaatttaactgt

SEQ ID NO: 100 - DNA, 21 bp, Homo sapiens
ggtcacccctgttcaggctct

SEQ ID NO: 101 - DNA, 21 bp, Homo sapiens
gggcaccctcagagcctgaac

SEQ ID NO: 102 - DNA, 21 bp, Homo sapiens
ccaggggccaaggagctattt

SEQ ID NO: 103 - DNA, 21 bp, Homo sapiens
gtttctaatcccagcctgggc

SEQ ID NO: 104 - DNA, 20 bp, Homo sapiens
agccacattcctgcccaggc

SEQ ID NO: 105 - DNA, 21 bp, Homo sapiens
attagaaacagaaacatttcg

SEQ ID NO: 106 - DNA, 21 bp, Homo sapiens
acagaaacatttcggggggtg

SEQ ID NO: 107 - DNA, 21 bp, Homo sapiens
ctcttggctctcggagccgca

SEQ ID NO: 108 - DNA, 21 bp, Homo sapiens
cctgcggctccgagagccaag

SEQ ID NO: 109 - DNA, 21 bp, Homo sapiens
gaaactaggaggcaaggaccg

SEQ ID NO: 110 - DNA, 21 bp, Homo sapiens
atgaggggccctggctgcggg

SEQ ID NO: 111 - DNA, 21 bp, Homo sapiens
gcaggatgctcttctccccaa

SEQ ID NO: 112 - DNA, 21 bp, Homo sapiens
ggtcctgctccagccgctggt

SEQ ID NO: 113 - DNA, 21 bp, Homo sapiens
ccggcttttcctgctatgaaa

SEQ ID NO: 114 - DNA, 21 bp, Homo sapiens
gcagtccgctttccccagagg

SEQ ID NO: 115 - DNA, 21 bp, Homo sapiens
aaagcggactgcctagaacca

SEQ ID NO: 116 - DNA, 21 bp, Homo sapiens
ttgctattgtctgtgcttttg

SEQ ID NO: 117 - DNA, 21 bp, Homo sapiens
acagacaatagcaagggcagc

SEQ ID NO: 118 - DNA, 21 bp, Homo sapiens
gaccagtcacatgctggggac

SEQ ID NO: 119 - DNA, 21 bp, Homo sapiens
tcacatgctggggacagggat

SEQ ID NO: 120 - DNA, 21 bp, Homo sapiens
atggctaggaggctgggggca

SEQ ID NO: 121 - DNA, 19 bp, Homo sapiens
ggcacagtggtgtgaagcc

SEQ ID NO: 122 - DNA, 21 bp, Homo sapiens
gtcaaggggagggagccagcc

SEQ ID NO: 123 - DNA, 21 bp, Homo sapiens
gctgccaccctggccttgccc

SEQ ID NO: 124 - DNA, 21 bp, Homo sapiens
tgagttctttccccagcacct

SEQ ID NO: 125 - DNA, 21 bp, Homo sapiens
gtggtagaggtaggactgtcc

SEQ ID NO: 126 - DNA, 21 bp, Homo sapiens
cctacctctaccactgacttg

SEQ ID NO: 127 - DNA, 89 bp, Homo sapiens
gtggtgggggaaaagaaccgagggtcaccagaaagggcactggagacaagggggaaagccccaccctctgtctgtctccccttctctct

SEQ ID NO: 128 - DNA, 25 bp, Homo sapiens
aagtggaggcaggatggagagaggg

SEQ ID NO: 129 - DNA, 317 bp, Homo sapiens
cccggtctcagtttaccctgccaacccaacttcatcctctactaaagggaggccaggagagtcattaggggctgtgggaggttgggagaaggtttttccagaggctgaatggcctggcccttcttggcctccaccttcccatctgtgaaatgggaggaggagggaaaggaaccaggcctgggtcagaaagagatggacacaagaggaagcaggggaaggcggagaaacaggaaacaagtgtcacacacagtttgttacacacaggccaaaaaccaaacaccagtcactgagggccagacatatgagggccagagggg

SEQ ID NO: 130 - DNA, 243 bp, Homo sapiens
agaaaccagtgaccagtgagcccgagtcctgggcttggggaggaggtgggcagacaaggcagctggcagaagcggaagcatcctccttccagccctggccccaggccctggaaagcccttgcaagaaggggttttgtgggacagctggaaggttggaggccctgggccaggagaactaaaggacgcagggacccggggtggcgggaatggtggctgacccacacggctcataggaaccgagac

SEQ ID NO: 131 - DNA, 61 bp, Homo sapiens
tgtgactcctaagccatggctctgagtcacagccaccctagcctctgctgaccttactctg

SEQ ID NO: 132 - DNA, 57 bp, Homo sapiens
accccatctgcaggagccccgagggtaggcactcaccccactgcaagaagtcggcca

SEQ ID NO: 133 - DNA, 189 bp, Homo sapiens
gggaggccagggcagcttcttgggtgactcagagatggattccgactcggacagttaaatttagccctcaggctctctgctttataccagctttttttttttttttttttttttttcccaggaggtggggaaggggtggtgaggacaggaccaggagggggcaccctcagagcctgaacaggggtgacc

SEQ ID NO: 134 - DNA, 40 bp, Homo sapiens
gcccaggctgggattagaaacagaaacatttcggggggtg

SEQ ID NO: 135 - DNA, 28 bp, Homo sapiens
attagaaacagaaacatttcggggggtg

SEQ ID NO: 136 - DNA, 106 bp, Homo sapiens
agaaaccagtgaccagtgagcccgagtcctgggcttggggaggaggtgggcagacaaggcagctggcagaagcggaagcatcctccttccagccctggccccaggc

SEQ ID NO: 137 - DNA, 363 bp, Homo sapiens
agccacattcctgcccaggctgggattagaaacagaaacatttcggggggtggagggtggcgcgggaagacacactccctgcggctccgagagccaagaggaatctgagctcttctttccagggtggacggttctccagagtggaagtcggggcctccaaccggcctggggtgcctgggccttgagagggccaggcctgagaaactaggaggcaaggaccgaggagtcccagctgggcagggcctgggaaacgtggtcctgggcagttctgataatttaaaaaacaccgaggactttgatgaggggccctggctgcgggcaggatgctcttctccccaagagggtcctgctccagccgctggt

SEQ ID NO: 138 - RNA, 19 nt, Francisella novicid
  misc_feature (1)..(19): 5′-handle of crRNA
aauuucuacuguuguagau

SEQ ID NO: 139 - protein, 1053 aa, Staphylococcus aureus
  VARIANT (10)..(10): conversion of Asp residue into Ala residue
  VARIANT (580)..(580): conversion of Asn residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 140 - protein, 1053 aa, Staphylococcus aureus
  VARIANT (10)..(10): conversion of Asp residue into Ala residue
  VARIANT (557)..(557): conversion of His residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 141 - protein, 1028 aa, Artificial Sequence
  amino acid residues  (721st to 745th amino acid residues of dSaCas9) deletion mutant
  VARIANT (10)..(10): conversion of Asp residue into Ala residue
  VARIANT (580)..(580): conversion of Asn residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKTEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 142 - protein, 6 aa, Artificial Sequence
  GGSGGS linker
GGSGGS

SEQ ID NO: 143 - protein, 1034 aa, Artificial Sequence
  amino acid residues  (721st to 745th amino acid residues of dSaCas9) deletion mutant with GGSGGS linker
  VARIANT (10)..(10): conversion of Asp residue into Ala residue
  VARIANT (580)..(580): conversion of Asn residue into Ala residue
  MISC_FEATURE (721)..(726): GGSGGS linker
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKGGSGGSTEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 144 - protein, 886 aa, Artificial Sequence
  amino acid residues  (482nd to 648th amino acid residues of dSaCas9) deletion mutant
  VARIANT (10)..(10): conversion of Asp residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELARETRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 145 - protein, 892 aa, Artificial Sequence
  amino acid residues  (482nd to 648th amino acid residues of dSaCas9) deletion mutant with GGSGGS linker
  VARIANT (10)..(10): conversion of Asp residue into Ala residue
  MISC_FEATURE (482)..(487): GGSGGS linker
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREGGSGGSTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 146 - RNA, 21 nt, Homo sapiens
  misc_feature (1)..(21): crRNA corresponding to the target sequence (SEQ ID NO5)
cccagucgaggccaaagaaga

SEQ ID NO: 147 - DNA, 21 bp, Homo sapiens
  misc_feature (1)..(21): sequence comprementary to the target sequence (SEQ ID No5)
tcttctttggcctcgactggg

SEQ ID NO: 148 - DNA, 19 bp, Francisella novicid
  misc_feature: 5′-handle of crRNA
aatttctactgttgtagat

SEQ ID NO: 149 - DNA, 83 bp, Staphylococcus aureus
  misc_feature (1)..(83): sequence encoding tracrRNA
gttttagtactctggaaacagaatctactaaaacaaggcaaaatgccgtgtttatctcgtcaacttgttggcgagattttttt

SEQ ID NO: 150 - DNA, 76 bp, Staphylococcus aureus
  misc_feature (1)..(76): SaCas9 gRNA scaffold sequence
gttttagtactctggaaacagaatctactaaaacaaggcaaaatgccgtgtttatctcgtcaacttgttggcgaga

SEQ ID NO: 151 - DNA, 3477 bp, Artificial Sequence
  dSaCas9 fused to KRAB (DNA)
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 152 - protein, 1159 aa, Artificial Sequence
  dSaCas9 fused to KRAB (Protein)
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSMDAKSLTAWSRTLVTFKDVFVDFTREEWKLLDTAQQIVYRNVMLENYKNLVSLGYQLTKPDVILRLEKGEEP

SEQ ID NO: 153 - DNA, 399 bp, Artificial Sequence
  blastR cassette (DNA)
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 154 - protein, 132 aa, Artificial Sequence
  blastR cassette (Protein)
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 155 - DNA, 241 bp, Artificial Sequence
  U6 promoter
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 156 - RNA, 82 nt, Staphylococcus aureus
  misc_feature (1)..(82): tracrRNA
guuuuaguacucuggaaacagaaucuacuaaaacaaggcaaaaugccguguuuaucacgucaacuuguuggcgagauuuuuu

SEQ ID NO: 157 - RNA, 19 nt, Homo sapiens
guggugggggaaaagaacc

SEQ ID NO: 158 - RNA, 21 nt, Homo sapiens
agagagaaggggagacagaca

SEQ ID NO: 159 - RNA, 21 nt, Homo sapiens
gggugugcaggaugguuaggg

SEQ ID NO: 160 - RNA, 21 nt, Homo sapiens
aaguggaggcaggauggagag

SEQ ID NO: 161 - RNA, 21 nt, Homo sapiens
ggaggcaggauggagagaggg

SEQ ID NO: 162 - RNA, 21 nt, Homo sapiens
gcaggguaaacugagaccggg

SEQ ID NO: 163 - RNA, 21 nt, Homo sapiens
gacauaugagggccagagggg

SEQ ID NO: 164 - RNA, 21 nt, Homo sapiens
agaaaccagugaccagugagc

SEQ ID NO: 165 - RNA, 21 nt, Homo sapiens
gccuggggccagggcuggaag

SEQ ID NO: 166 - RNA, 21 nt, Homo sapiens
ucagccaccauucccgccacc

SEQ ID NO: 167 - RNA, 21 nt, Homo sapiens
gucucgguuccuaugagccgu

SEQ ID NO: 168 - RNA, 21 nt, Homo sapiens
ugugacucagagccauggcuu

SEQ ID NO: 169 - RNA, 21 nt, Homo sapiens
ugugacuccuaagccauggcu

SEQ ID NO: 170 - RNA, 19 nt, Homo sapiens
ugacucagagccauggcuu

SEQ ID NO: 171 - RNA, 21 nt, Homo sapiens
cagaguaaggucagcagaggc

SEQ ID NO: 172 - RNA, 19 nt, Homo sapiens
gggcuccugcagauggggu

SEQ ID NO: 173 - RNA, 21 nt, Homo sapiens
uggccgacuucuugcaguggg

SEQ ID NO: 174 - RNA, 21 nt, Homo sapiens
ggggcuccagccccaggaagc

SEQ ID NO: 175 - RNA, 21 nt, Homo sapiens
auucccccuacucuagcacug

SEQ ID NO: 176 - RNA, 21 nt, Homo sapiens
agcuucuugggugacucagag

SEQ ID NO: 177 - RNA, 21 nt, Homo sapiens
cugagggcuaaauuuaacugu

SEQ ID NO: 178 - RNA, 21 nt, Homo sapiens
ggucaccccuguucaggcucu

SEQ ID NO: 179 - RNA, 21 nt, Homo sapiens
guuucuaaucccagccugggc

SEQ ID NO: 180 - RNA, 21 nt, Homo sapiens
auuagaaacagaaacauuucg

SEQ ID NO: 181 - RNA, 21 nt, Homo sapiens
acagaaacauuucggggggug

SEQ ID NO: 182 - RNA, 21 nt, Homo sapiens
ccugcggcuccgagagccaag

SEQ ID NO: 183 - RNA, 21 nt, Homo sapiens
gaaacuaggaggcaaggaccg

SEQ ID NO: 184 - RNA, 21 nt, Homo sapiens
gcaggaugcucuucuccccaa

SEQ ID NO: 185 - RNA, 21 nt, Homo sapiens
acagacaauagcaagggcagc

SEQ ID NO: 186 - RNA, 21 nt, Homo sapiens
ucacaugcuggggacagggau

SEQ ID NO: 187 - DNA, 450 bp, Artificial Sequence
  CK8 promoter
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 188 - protein, 16 aa, Artificial Sequence
  Nuclear localization signal
APKKKRKVGIHGVPAA

SEQ ID NO: 189 - DNA, 48 bp, Artificial Sequence
  DNA sequence encoding NLS of SEQ ID NO 188
gccccaaagaagaagcggaaggtcggtatccacggagtcccagcagcc

SEQ ID NO: 190 - protein, 16 aa, Artificial Sequence
  Nuclear localization signal
KRPAATKKAGQAKKKK

SEQ ID NO: 191 - DNA, 48 bp, Artificial Sequence
  DNA sequence encoding NLS of SEQ ID NO 190
aaaaggccggcggccacgaaaaaggccggccaggcaaaaaagaaaaag

SEQ ID NO: 192 - DNA, 20 bp, Artificial Sequence
  DMD exon 78 forward primer
ttagaggaggtgatggagca

SEQ ID NO: 193 - DNA, 20 bp, Artificial Sequence
  DMD exon 78 reverse primer
gatactaaggactccatcgc

SEQ ID NO: 194 - DNA, 21 bp, Artificial Sequence
  MBNL1 exon 7 forward primer
gctgcccaataccaggtcaac

SEQ ID NO: 195 - DNA, 22 bp, Artificial Sequence
  MBNL1 exon 7 reverse primer
tggtgggagaaatgctgtatgc

SEQ ID NO: 196 - DNA, 23 bp, Artificial Sequence
  KIF13A exon 21 forward primer
acctgtgcagcattcagggacac

SEQ ID NO: 197 - DNA, 24 bp, Artificial Sequence
  KIF13A exon 21 reverse primer
ctcgtcgtttaatgagtgcatctg

SEQ ID NO: 198 - DNA, 24 bp, Artificial Sequence
  TNNT2 exon 5 forward primer
atagaagaggtggtggaagagtac

SEQ ID NO: 199 - DNA, 24 bp, Artificial Sequence
  TNNT2 exon 5 reverse primer
gtctcagcctctgcttcagcatcc

SEQ ID NO: 200 - DNA, 3480 bp, Artificial Sequence
  dSaCas9 fused to KRAB (DNA) with terminal stop codon
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctga

SEQ ID NO: 201 - DNA, 208 bp, Artificial Sequence
  bGlobin polyA sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 202 - DNA, 242 bp, Artificial Sequence
  U6 polymer
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacg

SEQ ID NO: 203 - DNA, 8241 bp, Artificial Sequence
  pED0001
gatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccggaagcggtgctactaacttcagcctgctgaagcaggctggagacgtggaggagaaccctggacctaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaag

SEQ ID NO: 204 - DNA, 282 bp, Artificial Sequence
  EFS promoter
ctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgcca